(19) United States
(12) Patent Application Publication
(10) Pub. No.: US 2016/0168147 A1
(43) Pub. Date: Jun. 16, 2016

(54) ISOTOPICALLY ENRICHED AZAINDOLES

(71) Applicant: Vertex Pharmaceuticals Incorporated, Boston, MA (US)

(72) Inventors: Christopher Lee Brummel, Marlborough, MA (US); Francois Maltais, Tewksbury, MA (US); David D. Deininger, Waltham, MA (US); Brian Ledford, Norton, MA (US); Warren Dorsch, Waltham, MA (US)

(21) Appl. No.: 15/049,812

(22) Filed: Feb. 22, 2016

Related U.S. Application Data
(63) Continuation of application No. PCT/US2014/051988, filed on Aug. 21, 2014.
(60) Provisional application No. 61/943,721, filed on Feb. 24, 2014.
(60) Provisional application No. 61/868,703, filed on Aug. 22, 2013.
(60) Provisional application No. 61/868,770, filed on Aug. 22, 2013.

(51) Int. Cl. C07D 471/04; C07D 239/42; C07F 5/02

(52) U.S. Cl. CPC C07D 471/04; C07F 5/025; C07D 239/42

(57) ABSTRACT

The present invention relates to deuterated compounds that are useful for inhibiting Janus kinases and processes and intermediates useful for preparing such compounds.

### CROSS REFERENCE TO RELATED APPLICATION

This PCT application claims the benefit of U.S. provisional application Ser. Nos. 61/868,770 and 61/868,703, which were both filed on Aug. 22, 2014, and U.S. provisional application serial no. 61/943,721, which was filed on Feb. 24, 2014. Each of these documents is hereby incorporated by reference in its entirety.

### TECHNICAL FIELD OF THE INVENTION

The present invention relates to a compound useful as an inhibitor of Janus kinases (JAKs) as well as processes and intermediates for the preparation of the compound.

### BACKGROUND OF THE INVENTION

The Janus kinases (JAK) are a family of tyrosine kinases consisting of JAK1, JAK2, JAK3 and TYK2. The JAKs play a critical role in cytokine signaling. The down-stream substrates of the JAK family of kinases include the signal transducer and activator of transcription (STAT) proteins. JAK/STAT signaling has been implicated in the mediation of many abnormal immune responses such as allergies, asthma, transplant rejection, rheumatoid arthritis, amyotrophic lateral sclerosis and multiple sclerosis as well as in solid and hematologic malignancies such as leukemias and lymphomas. JAK2 has also been implicated in myeloproliferative disorders, which include polycythemia vera, essential thrombocythemia, chronic idiopathic myelofibrosis, myeloid metaplasia with myelofibrosis, chronic myeloid leukemia, chronic myelomonocytic leukemia, chronic eosinophilic leukemia, hypereosinophilic syndrome and systematic mast cell disease.

Compounds described as kinase inhibitors, particularly the JAK family kinases, are disclosed in WO 2005/095400, WO 2007/084557, and WO 2013/006634. The entire contents of these PCT publications are incorporated herein by reference. Also disclosed in these publications are processes and intermediates for the preparation of these compounds.

Substitution of deuterium for hydrogen on the azaindole ring system of the compound 2-((2-(1H-pyrrolo[2,3-b]pyridin-3-yl)pyrimidin-4-y0amino)-2-methyl-N-(2,2,2-trifluoroethypbutanamide results in a slower rate of oxidation of the C-D bond relative to the rate of oxidation of corresponding C—H bond in the non-deuterated compound. This isotopic effect acts to reduce formation of metabolites and thereby alters the pharmacokinetic parameters of the compound. Lower rates of oxidation, metabolism, and clearance result in greater and more sustained biological activity. Deuteration is targeted at various sites (e.g., the C2 site) of the compound to increase the potency of drug, reduce toxicity of the drug, reduce the clearance of the pharmacologically active compound, and improve the stability of the molecule.

### SUMMARY OF THE INVENTION

The present invention relates to a compound useful as a JAK inhibitor and processes for generating the compound.

The present invention provides a compound of Formula I:

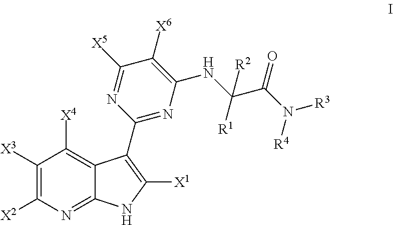

or a pharmaceutically acceptable salt thereof, wherein each of X¹, X², X³, X⁴, X⁵, and X⁶ is independently —H or -D; R¹ is —C₁₋₄ alkyl having 0 to 3 hydrogen atoms replaced with deuterium atoms; R² is —C₂₋₄ alkyl having 0 to 7 hydrogen atoms replaced with deuterium atoms; R³ is —H or unsubstituted —C₁₋₂ alkyl; and R⁴ is —CH₂CR₃ or —(CH₂)₂CR₃ wherein each R is independently —H or —F; provided that i) at least one of X¹, X², X³, X⁴, X⁵, and X⁶ is -D, or at least one of R¹ and R² has at least 1 hydrogen atom that is replaced with a deuterium atom; and ii) when X⁵ is -D, then X⁶ is -D or X² is —H.

In some embodiments, at least one of X¹, X², X³, and X⁴ is -D. For example, at least two of X¹, X², X³, and X⁴ is -D. In some instances, at least three of X¹, X², X³, and X⁴ is -D. In other instances, each of X¹, X², X³, and X⁴ is -D.

In some embodiments, X¹ is -D.

In some embodiments, R¹ is methyl having 1 to 3 hydrogen atoms replaced with deuterium atoms. For example, R¹ is methyl having 3 hydrogen atoms replaced with deuterium atoms.

In some embodiments, R¹ is ethyl having 1 to 5 hydrogen atoms replaced with deuterium atoms. For example, R¹ is ethyl having 5 hydrogen atoms replaced with deuterium atoms.

In some embodiments, R² is propyl having 1 to 7 hydrogen atoms replaced with deuterium atoms. For example, R² is propyl having 7 hydrogen atoms replaced with deuterium atoms.

And, in some embodiments, R¹ is methyl having 3 hydrogen atoms replaced with deuterium atoms, and R² is ethyl having 5 hydrogen atoms replaced with deuterium atoms.

In some embodiments, X⁵ and X⁶ are each -D.

In some embodiments, R³ is —H.

In some embodiments, R⁴ is —CH₂CF₃.

In some embodiments, the compound of Formula I is a compound in Table 1.

Another aspect of the present invention provides a compound of Formula II:

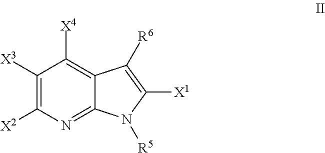

or a pharmaceutically acceptable salt thereof, wherein each of X¹, , X², X³, and X⁴ is independently —H or -D; R⁵ is —H or —PG¹ , wherein —PG¹ is an amine protecting group; and R⁶ is —H, halo, or —B(OR⁷)₂, wherein each R⁷ is independently —H, —C₁₋₄ alkyl, or two —OR⁷ groups taken together with the boron atom to which they are attached form a 5-6 membered heterocycle optionally substituted with 1-4 C₁₋₃ alkyl groups.

In some embodiments, R⁵ is PG¹, and PG¹ is SO₂phenyl. In other embodiments, PG¹ is a tosyl or Boc protecting group.

In some embodiments, at least one of X¹, X², X³, and X⁴ is -D. For example, at least two of X¹, X², X³, and X⁴ is -D. In some instances, at least three of X¹, X², X³, and X⁴ is -D. In other instances, each of X¹, X², X³, and X⁴ is -D.

In some embodiments, X¹, is -D.

Another aspect of the present invention provides a compound of Formula III:

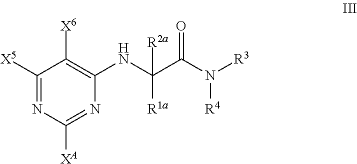

or a pharmaceutically acceptable salt thereof, wherein each of X⁵ and X⁶ is —H or -D; X^{A} is a leaving group; R^{1a} is —C₁₋₄ alkyl having 1 to 3 hydrogen atoms replaced with deuterium atoms; R^{2a} is C₂₋₄ alkyl having 1 to 7 hydrogen atoms replaced with deuterium atoms; R³ is —H or unsubstituted —C₁₋₂ alkyl; and R⁴ is —CH₂CR₃ or —(CH₂)₂CR₃ wherein each R is independently —H or —F.

In some embodiments, X^{1a} is halo. For example, X^{A} is —Cl or —Br.

In some embodiments, R^{1a} is methyl having 1 to 3 hydrogen atoms replaced with deuterium atoms. For example, R^{1a} is methyl having 3 hydrogen atoms replaced with deuterium atoms.

In some embodiments, R^{1a} is ethyl having 1 to 5 hydrogen atoms replaced with deuterium atoms. For example, R^{1a} is ethyl having 5 hydrogen atoms replaced with deuterium atoms.

In some embodiments, R^{2a} is ethyl having 1 to 5 hydrogen atoms replaced with deuterium atoms. For example, R^{2a} is ethyl having 5 hydrogen atoms replaced with deuterium atoms.

In some embodiments, R^{2a} is propyl having 1 to 7 hydrogen atoms replaced with deuterium atoms. For example, R^{2a} is propyl having 7 hydrogen atoms replaced with deuterium atoms.

In some embodiments, R³ is —H.

In some embodiments, R⁴ is —CH₂CF₃.

The present invention provides a compound of Formula I-e:

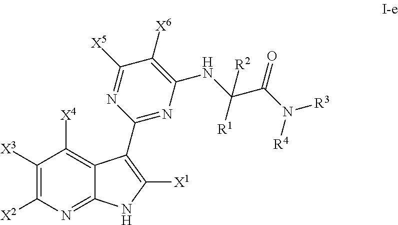

or a pharmaceutically acceptable salt thereof, wherein each of X¹, X², X³, X⁴, X⁵, and X⁶ is independently —H or -D; R¹ is C₁₋₄ alkyl having 0 to 3 hydrogen atoms replaced with deuterium atoms; R² is —C₂₋₄ alkyl having 0 to 7 hydrogen atoms replaced with deuterium atoms; R³ is —H or unsubstituted C₁₋₂ alkyl; and R⁴ is —CH₂CR₃ or —(CH₂)₂CR₃ wherein each R is independently —H or —F; provided that i) the total number of deuterium atoms on the compound of Formula I is at least two; and ii) when X⁵ is -D, then X⁶ is -D or X² is —H.

Another aspect of the present invention provides a process for preparing a compound of Formula I:

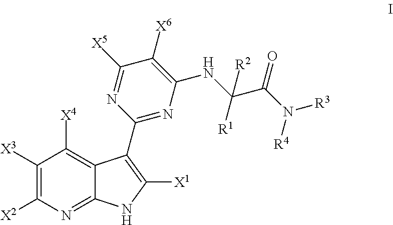

or a pharmaceutically acceptable salt thereof, wherein each of X¹, X², X³, X⁴, X⁵, and X⁶ is independently H or -D; R¹ is —C₁₋₄ alkyl having 0 to 3 hydrogen atoms replaced with deuterium atoms; R² is —C₂₋₄ alkyl having 0 to 7 hydrogen atoms replaced with deuterium atoms; R³ is H or unsubstituted —C₁₋₂ alkyl; and R⁴ is —CH₂CR₃ or —(CH₂)₂CR₃ wherein each R is independently —H or —F; provided that i) at least one of X¹, X², X³, X⁴, X⁵, and X⁶ is -D, or at least one of R¹ and R² has at least 1 hydrogen atom that is replaced with a deuterium atom; and ii) when X⁵ is -D, then X⁶ is -D or X² is —H, comprising the steps of

a) reacting a compound of Formula 1, wherein each R⁷ is independently —H, —C₁₋₄ alkyl, or two —OR⁷ groups taken together with the boron atom to which they are attached form a 5-6 membered heterocycle optionally substituted with 1-4 C₁₋₃ alkyl groups, and PG¹ is an amine protecting group, with a compound of Formula 2, wherein X^{A} is a leaving group,

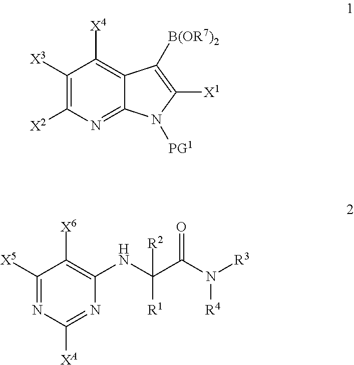

in the presence of a base and a palladium catalyst to generate a compound of Formula 3, and

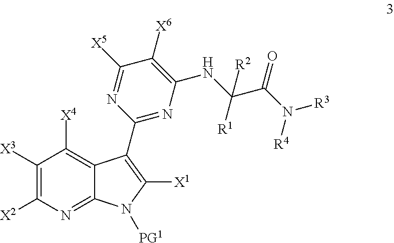

b) deprotecting the compound of Formula 3 to generate the compound of Formula I.

In still another aspect, the present invention provides a process for preparing a compound of Formula I:

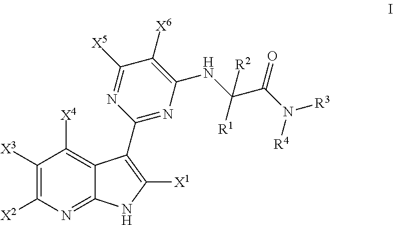

or a pharmaceutically acceptable salt thereof, wherein each of X¹, X², X³, X⁴, X⁵, and X⁶ is independently —H or -D; R¹ is C₁₋₄ alkyl having 0 to 3 hydrogen atoms replaced with deuterium atoms; R² is C₂₋₄ alkyl having 0 to 7 hydrogen atoms replaced with deuterium atoms; R³ is —H or unsubstituted C₁₋₂ alkyl; and R⁴ is CH₂CR₃ or —(CH₂)₂CR₃ wherein each R is independently —H or —F; provided that i) the compound of Formula I has at least two deuteriums; and ii) when X⁵ is -D, then X⁶ is D or X² is —H, comprising the steps of:

a) reacting a compound of Formula 1, wherein each R⁷ is independently —H, C₁₋₄ alkyl, or two —OR⁷ groups taken together with the boron atom to which they are attached form a 5-6 membered heterocycle optionally substituted with 1-4 C₁₋₃ alkyl groups, and PG¹ is an amine protecting group, with a compound of Formula 2, wherein X^{A} is a leaving group,

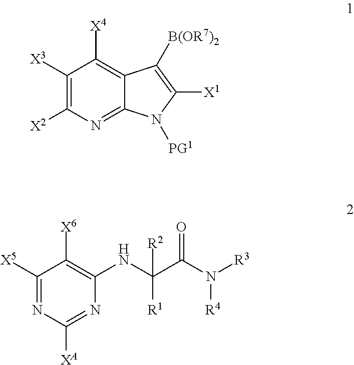

in the presence of a base and a palladium catalyst to generate a compound of Formula 3, and

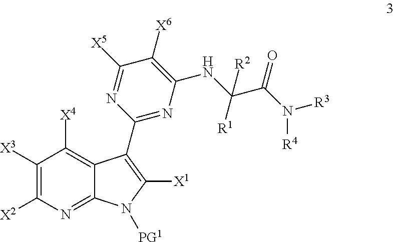

b) deprotecting the compound of Formula 3 to generate the compound of Formula I.

In some embodiments, X^{A} is halo. For example, X^{A} is —Cl or —Br.

Some embodiments further comprise step c) reacting a compound of Formula 4:

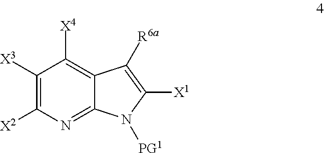

wherein R^{6a} is a leaving group, with a borylating agent to generate the compound of Formula 1.

In some embodiments, R^{6a} is a halogen. For instance, R6a is —Cl, —Br, or —I.

In some embodiments, the borylating agent comprises bis-pinacol borane. For example, the borylaying agent comprises 2-isopropoxy-4,4,5,5-tetramethyl-1,3,2-dioxaborolane.

Some embodiments further comprise step d) reacting a compound of Formula 5:

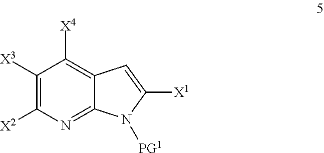

with R^{6a}—X^{B}, wherein X^{B} is halo, in the presence of an organic solvent to generate the compound of Formula 4.

In some embodiments, R^{6a}—X^{B} is Br₂.

Some embodiments further comprise steps e) protecting the compound of Formula 6:

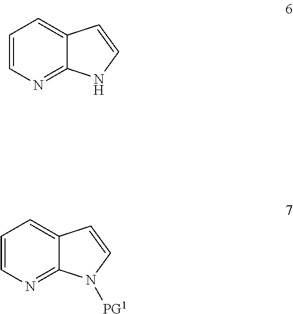

with amine protecting group PG¹, to generate the compound of Formula 7 and

f) reacting the compound of Formula 7 with a deuterating agent to generate the compound of Formula 5.

In some embodiments, PG¹ is —SO₂-phenyl. In other embodiments, —PG¹ is a tosyl or Boc protecting group.

### BRIEF DESCRIPTION OF THE DRAWING

The following figures are provided by way of example and are not intended to limit the scope of the invention.

FIG. 1A is an HPLC chromatograph for the assay of Compound A, i.e., the native compound, as described in Example 6.

FIG. 1B is an HPLC chromatograph for the assay of Compound 1-a as described in Example 6.

FIG. 1C is an HPLC chromatograph for the assay of Compound 4 as described in Example 6.

FIG. 1D is an HPLC chromatograph for a second assay of Compound 4 as described in Example 6.

FIG. 2A is an HPLC chromatograph for the assay of Compound A as described in Example 6.

FIG. 2B is an HPLC chromatograph for the assay of Compound 6 as described in Example 6.

FIG. 2C is an HPLC chromatograph for the assay of Compound 8 as described in Example 6.

FIG. 2D is an HPLC chromatograph for the assay of Compound 9 as described in Example 6.

FIG. 3A is an HPLC chromatograph for the assay of Compound A as described in Example 6.

FIG. 3B is an HPLC chromatograph for the assay of Compound 7 as described in Example 6.

FIG. 3C is an HPLC chromatograph for the assay of Compound 3 as described in Example 6.

FIG. 3D is an HPLC chromatograph for the assay of Compound 2 as described in Example 6.

FIG. 4A is an LCMS chromatograph for the assay of the M9 metabolite of Compound A as described in Example 6.

FIG. 4B is an LCMS chromatograph for the assay of the M9 metabolite of Compound 8 as described in Example 6.

FIG. 4C is an LCMS chromatograph for the assay of the M9 metabolite of Compound 9 as described in Example 6.

FIG. 5A is an LCMS chromatograph for the assay of the M6 metabolite of Compound A as described in Example 6.

FIG. 5B is an LCMS chromatograph for the assay of the M6 metabolite of Compound 3 as described in Example 6.

FIG. 6 is a plot of concentration as a function of time for the formation of the Compound B (metabolite), from Compound A (native compound); and the formation of Compound B from Compound 1-a (deuterated compound), as described in Example 7.

### DETAILED DESCRIPTION OF THE INVENTION

The present invention provides a compound of Formula I:

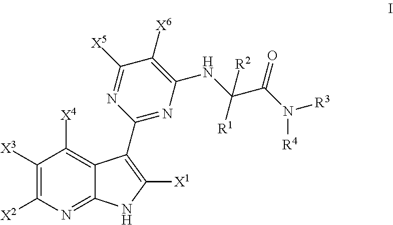

or a pharmaceutically acceptable salt thereof, wherein each of X¹, X², X³, X⁴, X⁵, and X⁶ is independently —H or -D; R¹ is C₁₋₄ alkyl having 0 to 3 hydrogen atoms replaced with deuterium atoms; R² is —C₂₋₄ alkyl having 0 to 7 hydrogen atoms replaced with deuterium atoms; R³ is —H or unsubstituted C₁₋₂ alkyl; and R⁴ is —CH₂CR₃ or —(CH₂)₂CR₃ wherein each R is independently —H or —F; provided that i) at least one of X¹, X², X³, X⁴, X⁵, and X⁶ is -D, or at least one of R¹ and R² has at least 1 hydrogen atom that is replaced with a deuterium atom; and ii) when X⁵ is -D, then X⁶ is D or X² is —H.

As used herein, the following definitions shall apply unless otherwise indicated.

### I. DEFINITIONS

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

As described herein, compounds of the invention may optionally be substituted with one or more substituents, such as are illustrated generally above, or as exemplified by particular classes, subclasses, and species of the invention.

As used herein, “D” refers to a deuterium radical.

As used herein, the terms “deuterium” and “D” are used interchangeably to refer to an isotope of hydrogen having one (1) proton and one (1) neutron.

As used herein, the term “hydroxyl” or “hydroxy” refers to an -OH moiety.

As used herein the term “aliphatic” encompasses the terms alkyl, alkenyl, alkynyl, each of which being optionally substituted as set forth below.

As used herein, an “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, or heterocycloaliphaticamino], sulfonyl [e.g., aliphatic-SO₂-], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains 2-8 (e.g., 2-12, 2-6, or 2-4) carbon atoms and at least one double bond. Like an alkyl group, an alkenyl group can be straight or branched. Examples of an alkenyl group include, but are not limited to allyl, 1- or 2-isopropenyl, 2-butenyl, and 2-hexenyl. An alkenyl group can be optionally substituted with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, heterocycloaliphaticamino, or aliphaticsulfonylamino], sulfonyl [e.g., alkyl-SO₂—, cycloaliphatic-SO₂—, or aryl-SO₂-], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-12, 2-6, or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl [e.g., aliphaticsulfanyl or eye loaliphaticsulfanyl], sulfinyl [e.g., aliphaticsulfinyl or cycloaliphaticsulfinyl], sulfonyl [e.g., aliphatic-SO₂—, aliphaticamino-SO₂—, or cycloaliphatic-SO₂—], amido [e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl], urea, thiourea, sulfamoyl, sulfamide, alkoxycarbonyl, alkylcarbonyloxy, cycloaliphatic, heterocycloaliphatic, aryl, heteroaryl, acyl [e.g., (cycloaliphatic)carbonyl or (heterocycloaliphatic)carbonyl], amino [e.g., aliphaticamino], sulfoxy, oxo, carboxy, carbamoyl, (cycloaliphatic)oxy, (heterocycloaliphatic)oxy, or (heteroaryl)alkoxy.

As used herein, an “amido” encompasses both “aminocarbonyl” and “carbonylamino”. These terms when used alone or in connection with another group refer to an amido group such as —N(R^{X})—C(O)—R^{Y} or —C(O)—N(R^{X})₂, when used terminally, and —C(O)—N(R^{X})— or —N(R^{X})—C(O)— when used internally, wherein R^{X} and R^{Y} can be aliphatic, cycloaliphatic, aryl, araliphatic, heterocycloaliphatic, heteroaryl or heteroaraliphatic. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen, aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—, where R^{X} has the same meaning as defined above.

As used herein, an “aryl” group used alone or as part of a larger moiety as in “aralkyl”, “aralkoxy”, or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphatic-SO₂— or amino-SO₂—]; sulfinyl [e.g., aliphatic-S(O)— or cycloaliphatic-S(O)—]; sulfanyl [e.g., aliphatic-S—]; cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl [e.g., mono-, di (such as p,m-dihaloaryl), and (trihalo)aryl]; (carboxy)aryl [e.g., (alkoxycarbonyl)aryl, ((aralkyl)carbonyloxy)aryl, and (alkoxycarbonyl)aryl]; (amido)aryl [e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl]; aminoaryl [e.g., ((alkylsulfonyl)amino)aryl or ((dialkyl)amino)aryl]; (cyanoalkyl)aryl; (alkoxy)aryl; (sulfamoyl)aryl [e.g., (aminosulfonyl)aryl]; (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic”, “alkyl”, and “aryl” are defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic [e.g., alkyl, alkenyl, or alkynyl, including carboxyalkyl, hydroxyalkyl, or haloalkyl such as trifluoromethyl], cycloaliphatic [e.g., cycloalkyl or cycloalkenyl], (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido [e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino], cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 6-12 (e.g., 8-12 or 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, bicyclo[2.2.2]octenyl, or bicyclo[3.3.1]nonenyl.

A cycloalkyl or cycloalkenyl group can be optionally substituted with one or more substituents such as phospho, aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl [e.g., alkyl-SO₂— and aryl-SO₂—], sulfinyl [e.g., alkyl-S(O)—], sulfanyl [e.g., alkyl-S—], sulfoxy, urea, thiourea, sulfamoyl, sulfamide, oxo, or carbamoyl.

As used herein, the term “heterocycloaliphatic” encompasses heterocycloalkyl groups and heterocycloalkenyl groups, each of which being optionally substituted as set forth below.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety to form structures, such as tetrahydroisoquinoline, which would be categorized as heteroaryls.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

A heterocycloalkyl or heterocycloalkenyl group can be optionally substituted with one or more substituents such as phospho, aliphatic [e.g., alkyl, alkenyl, or alkynyl], cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], nitro, cyano, halo, hydroxy, mercapto, sulfonyl [e.g., alkylsulfonyl or arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

A “heteroaryl” group, as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophene-yl, quinolinyl, or isoquinolinyl). Some examples of heteroaryl are azetidinyl, pyridyl, 1 H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophene-yl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphaticsulfonyl or aminosulfonyl]; sulfinyl [e.g., aliphaticsulfinyl]; sulfanyl [e.g., aliphaticsulfanyl]; nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl [e.g., mono- and di-(halo)heteroaryl]; (carboxy)heteroaryl [e.g., (alkoxycarbonyl)heteroaryl]; cyanoheteroaryl; aminoheteroaryl [e.g., ((alkylsulfonyl)amino)heteroaryl and ((dialkyl)amino)heteroaryl]; (amido)heteroaryl [e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyDamino)heteroaryl]; (cyanoalkyl)heteroaryl; (alkoxy)heteroaryl; (sulfamoyl)heteroaryl [e.g., (aminosulfonyl)heteroaryl]; (sulfonyl)heteroaryl [e.g., (alkylsulfonyl)heteroaryl]; (hydroxyalkyl)heteroaryl; (alkoxyalkyl)heteroaryl; (hydroxy)heteroaryl; ((carboxy)alkyl)heteroaryl; (((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl [e.g., (alkylcarbonyl)heteroaryl]; (alkyl)heteroaryl; or (haloalkyl)heteroaryl [e.g., trihaloalkylheteroaryl].

A “heteroaraliphatic (such as a heteroaralkyl group) as used herein, refers to an aliphatic group (e.g., a C₁₋₄ alkyl group) that is substituted with a heteroaryl group. “Aliphatic”, “alkyl”, and “heteroaryl” have been defined above.

A “heteroaralkyl” group, as used herein, refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “acyl” group refers to a formyl group or R^{X}—C(O)— (such as alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)-. The aryl and heteroaryl portion of the aroyl or heteroaroyl is optionally substituted as previously defined.

As used herein, an “alkoxy” group refers to an alkyl-O— group where “alkyl” has been defined previously.

As used herein, a “carbamoyl” group refers to a group having the structure —O—CO—NR^{X}R^{Y} or —NR^{X}—CO—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X}, when used as a terminal group; or —OC(O)— or —C(O)O— when used as an internal group.

As used herein, a “haloaliphatic” group refers to an aliphatic group substituted with 1-3 halogen. For instance, the term haloalkyl includes the group -CF₃.

As used herein, a “mercapto” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —O—S(O)₂—NR^{Y}R^{Z} wherein R^{Y} and R^{Z} have been defined above.

As used herein, a “sulfonamide” group refers to the structure —S(O)₂—NR^{X}R^{Y} or —NR^{X}—S(O)₂-R^{Z} when used terminally; or —S(O)₂—NR^{X}— or —NR^{X} —S(O)₂— when used internally, wherein R^{X}, R^{Y}, and R^{Z} are defined above.

As used herein a “sulfanyl” group refers to —S-R^{X} when used terminally and —S— when used internally, wherein R^{X} has been defined above. Examples of sulfanyls include aliphatic-S-, cycloaliphatic-S-, aryl-S-, or the like.

As used herein a “sulfinyl” group refers to —S(O)—R^{X} when used terminally and —S(O)— when used internally, wherein R^{X} has been defined above. Exemplary sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to —S(O)₂—R^{X} when used terminally and —S(O)₂— when used internally, wherein R^{X} has been defined above. Exemplary sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂— or the like.

As used herein, a “sulfoxy” group refers to —O—S(O)—R^{X} or —S(O)—O—R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl”, which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refers to —C(O)—.

As used herein, an “oxo” refers to ═O.

As used herein, the term “phospho” refers to phosphinates and phosphonates. Examples of phosphinates and phosphonates include —P(O)(R^{P})₂, wherein R^{P} is aliphatic, alkoxy, aryloxy, heteroaryloxy, (cycloaliphatic)oxy, (heterocycloaliphatic)oxy aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, the term “amidino” group refers to the structure —C═(NR^{X})N(R^{X}R^{Y}) wherein R^{X} and R^{Y} have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is 1-12. A branched aliphatic chain is a straight aliphatic chain that is substituted with one or more aliphatic groups. A branched aliphatic chain has the structure —[CQQ]ᵥ- where Q is independently a hydrogen or an aliphatic group; however, Q shall be an aliphatic group in at least one instance. The term aliphatic chain includes alkyl chains, alkenyl chains, and alkynyl chains, where alkyl, alkenyl, and alkynyl are defined above.

In general, the term “substituted”, whether preceded by the term “optionally” or not, refers to the replacement of hydrogen atoms in a given structure with the radical of a specified substituent or isotope. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970). As used herein, “patient” refers to a mammal, including a human.

Chemical structures and nomenclature are derived from ChemDraw, version 11.0.1, Cambridge, Mass.

It is noted that the use of the descriptors “first”, “second”, “third”, or the like is used to differentiate separate elements (e.g., solvents, reaction steps, processes, reagents, or the like) and may or may not refer to the relative order or relative chronology of the elements described.

As used herein, the term “pharmaceutically acceptable salt” refers to those salts which are, within the scope of sound medical judgement, suitable for use in contact with the tissues of humans and lower animals without undue toxicity, irritation, allergic response and the like.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As described herein, “protecting group” refers to a moiety or functionality that is introduced into a molecule by chemical modification of a functional group in order to obtain chemoselectivity in a subsequent chemical reaction. Standard protecting groups are provided in Wuts and Greene: “Greene's Protective Groups in Organic Synthesis” 4th Ed, Wuts, P.G.M. and Greene, T.W., Wiley-Interscience, New York:2006, which is incorporated herein by reference.

Examples of nitrogen protecting groups include acyl, aroyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl and chiral auxiliaries such as protected or unprotected D, L or D, L-amino acids such as alanine, leucine, phenylalanine and the like; sulfonyl groups such as benzenesulfonyl, p-toluenesulfonyl and the like; carbamate groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, a,a-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl and the like, arylalkyl groups such as benzyl, triphenylmethyl, benzyloxymethyl and the like and silyl groups such as trimethylsilyl and the like. Preferred N-protecting groups are benzenesulfonylchloride and the like.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

As used herein, the term “solvent” also includes mixtures of solvents.

### II. COMPOUNDS

The present invention provides a compound of Formula I:

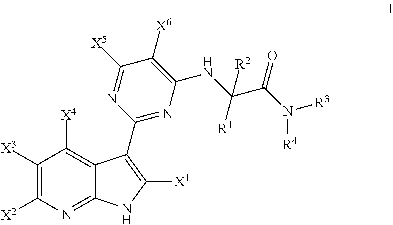

or a pharmaceutically acceptable salt thereof, wherein each of X¹, X², X³, X⁴, X⁵, and X⁶ is independently H or D; R¹ is C₁₋₄ alkyl having 0 to 3 hydrogen atoms replaced with deuterium atoms; R² is —C₂₋₄ alkyl having 0 to 7 hydrogen atoms replaced with deuterium atoms; R³ is —H or unsubstituted —C₁₋₂ alkyl; and R⁴ is —CH₂CR₃ or —(CH₂)₂CR₃ wherein each R is independently —H or —F; provided that i) at least one of X¹, X², X³, X⁴, X⁵, and X⁶ is -D, or at least one of R¹ and R² has at least 1 hydrogen atom that is replaced with a deuterium atom; and ii) when X⁵ is -D, then X⁶ is -D or X² is —H.

In some embodiments, when X¹ is -D, then at least one of X², X³, X⁴, X⁵, and X⁶ is also -D or at least one of the hydrogen atoms on R¹ or R² is replaced with D.

In some embodiments, at least one of X¹, X², X³, and X⁴ is -D. For example, at least two of X¹, X², X³, and X⁴ is D. In some instances, at least three of X¹, X², X³, and X⁴ is -D. In other instances, each of X¹, X², X³, and X⁴ is -D.

In some embodiments, X¹, is -D.

In some embodiments, X⁵ and X⁶ are -D.

In some embodiments, X⁵ is D and X² is —H.

In some embodiments, X⁵ and X⁶ are —H.

In some embodiments, R¹ is methyl.

In other embodiments, R¹ is methyl having 1 to 3 hydrogen atoms replaced with deuterium atoms. For example, R¹ is methyl having 3 hydrogen atoms replaced with deuterium atoms.

In some embodiments, R¹ is ethyl.

In other embodiments, R¹ is ethyl having 1 to 5 hydrogen atoms replaced with deuterium atoms. For example, R¹ is ethyl having 5 hydrogen atoms replaced with deuterium atoms.

In some embodiments, R² is ethyl.

In other embodiments, R² is ethyl having 1 to 5 hydrogen atoms replaced with deuterium atoms. For example, R² is ethyl having 5 hydrogen atoms replaced with deuterium atoms.

In some embodiments, R² is propyl.

In other embodiments, R² is propyl having 1 to 7 hydrogen atoms replaced with deuterium atoms. For example, R² is propyl having 7 hydrogen atoms replaced with deuterium atoms.

In some embodiments, R¹ is methyl and R² is ethyl.

In some embodiments, R¹ is methyl having 1-3 hydrogen atoms replaced with deuterium atoms and R² is ethyl.

In some embodiments, R¹ is methyl and R² is ethyl having 1-5 hydrogen atoms replaced with deuterium atoms.

In some embodiments, R¹ is methyl having 1-3 hydrogen atoms replaced with deuterium atoms and R² is ethyl having 1-5 hydrogen atoms replaced with deuterium atoms.

And, in some embodiments, R¹ is methyl having 3 hydrogen atoms replaced with deuterium atoms, and R² is ethyl having 5 hydrogen atoms replaced with deuterium atoms.

In some embodiments, R³ is H or methyl. For example, R³ is —H.

In some embodiments, R⁴ is —CH₂CF₃.

In some embodiments, the compound of Formula I is a compound of Formula I-a:

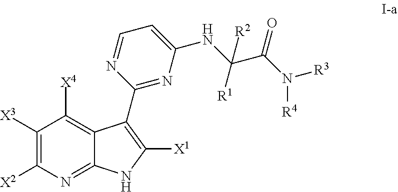

or a pharmaceutically acceptable salt thereof, wherein each of X¹, X², X³, and X⁴ is independently —H or -D; R¹ is —C₁₋₄ alkyl having 0 to 3 hydrogen atoms replaced with deuterium atoms; R² is C₂₋₄ alkyl having 0 to 7 hydrogen atoms replaced with deuterium atoms; R³ is —H or unsubstituted C₁₋₂ alkyl; and R⁴ is —CH₂CR₃ or —(CH₂)₂CR₃ wherein each R is independently —H or —F; wherein i) at least one of X¹, X², X³, and X⁴ is D; or ii) at least one of R¹ and R² has at least 1 hydrogen atom that is replaced with a deuterium atom.

In some embodiments, the compound of Formula I is a compound of Formula I-b:

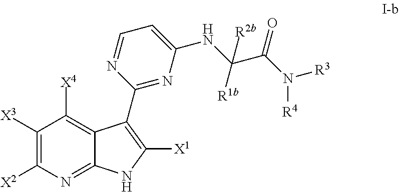

or a pharmaceutically acceptable salt thereof, wherein each of X¹, X², X³, and X⁴ is independently —H or -D; R^{1b} is —C₁₋₄ alkyl; R^{2b} is —C₂₋₄ alkyl; R³ is —H or unsubstituted —C₁₋₂ alkyl; and R⁴ is —CH₂CR₃ or —(CH₂)₂CR₃ wherein each R is independently —H or —F; wherein at least one of X¹, X², X³, and X⁴ is -D.

In some embodiments, the compound of Formula I is a compound of Formula I-c:

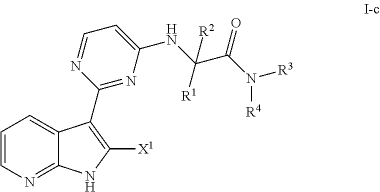

or a pharmaceutically acceptable salt thereof, wherein X¹ is independently H or -D; R¹ is —C₁₋₄ alkyl having 0-3 hydrogen atoms replaced with deuterium atoms; R² is —C₂₋₄ alkyl having 0-7 hydrogen atoms replaced with deuterium atoms; R³ is —H or unsubstituted —C₁₋₂ alkyl; and R⁴ is —CH₂CR₃ or —(CH₂)₂CR₃ wherein each R is independently —H or —F; wherein i) when X¹ is -D, then at least one of R¹ and R² has at least 1 hydrogen atom that is replaced with a deuterium atom.

In some embodiments, the compound of Formula I is a compound of Formula I-d:

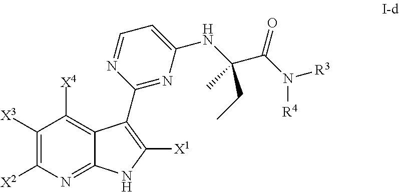

or a pharmaceutically acceptable salt thereof, wherein each of X¹, X², X³, and X⁴ is independently —H or -D; R³ is —H or unsubstituted —C₁₋₂ alkyl; and R⁴ is —CH₂CR₃ or —(CH₂)₂CR₃ wherein each R is independently —H or —F; wherein the compound of Formula I-d includes at least two D atoms.

The present invention provides a compound of Formula I-e:

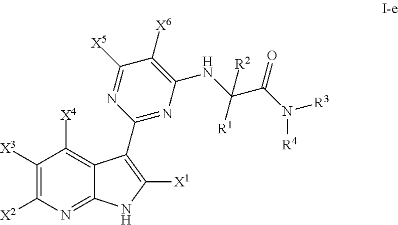

or a pharmaceutically acceptable salt thereof, wherein each of X¹, X², X³, X⁴, X⁵, and X⁶ is independently —H or -D; R¹ is C₁₋₄ alkyl having 0 to 3 hydrogen atoms replaced with deuterium atoms; R² is —C₂₋₄ alkyl having 0 to 7 hydrogen atoms replaced with deuterium atoms; R³ is —H or unsubstituted —C₁₋₂ alkyl; and R⁴ is —CH₂CR₃ or —(CH₂)₂CR₃ wherein each R is independently —H or —F; provided that i) the total number of deuterium atoms on the compound of Formula I is at least two; and ii) when X⁵ is -D, then X⁶ is -D.

In some embodiments, at least two of X¹, X², X³, and X⁴ is -D. In some instances, at least three of X¹, X², X³, and X⁴ is -D. In other instances, each of X¹, X², X³, and X⁴ is -D.

In some embodiments, X¹ is -D.

In some embodiments, X⁵ and X⁶ are -D.

In some embodiments, X⁵ and X⁶ are —H.

In some embodiments, R¹ is methyl.

In other embodiments, R¹ is methyl having 1 to 3 hydrogen atoms replaced with deuterium atoms. For example, R¹ is methyl having 3 hydrogen atoms replaced with deuterium atoms.

In some embodiments, R¹ is ethyl.

In other embodiments, R¹ is ethyl having 1 to 5 hydrogen atoms replaced with deuterium atoms. For example, R¹ is ethyl having 5 hydrogen atoms replaced with deuterium atoms.

In some embodiments, R² is ethyl.

In other embodiments, R² is ethyl having 1 to 5 hydrogen atoms replaced with deuterium atoms. For example, R² is ethyl having 5 hydrogen atoms replaced with deuterium atoms.

In some embodiments, R² is propyl.

In other embodiments, R² is propyl having 1 to 7 hydrogen atoms replaced with deuterium atoms. For example, R² is propyl having 7 hydrogen atoms replaced with deuterium atoms.

In some embodiments, R¹ is methyl and R² is ethyl.

In some embodiments, R¹ is methyl having 1-3 hydrogen atoms replaced with deuterium atoms and R² is ethyl.

In some embodiments, R¹ is methyl and R² is ethyl having 1-5 hydrogen atoms replaced with deuterium atoms.

In some embodiments, R¹ is methyl having 1-3 hydrogen atoms replaced with deuterium atoms and R² is ethyl having 1-5 hydrogen atoms replaced with deuterium atoms.

In some embodiments, R¹ is methyl having 3 hydrogen atoms replaced with deuterium atoms, and R² is ethyl having 5 hydrogen atoms replaced with deuterium atoms.

In some embodiments, R³ is —H or methyl. For example, R³ is —H.

In some embodiments, R⁴ is —CH₂CF₃.

The present invention provides a compound of Formula I-f:

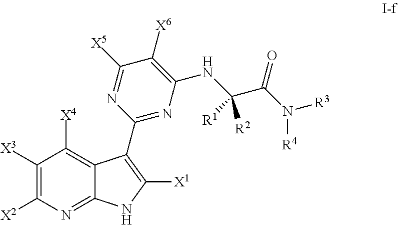

or a pharmaceutically acceptable salt thereof, wherein R¹ is —C(Y¹)(Y²)(Y³); R² is —C(Z¹)(Z²)—C(Z³)(Z⁴)(Z⁵); R³ is —H or unsubstituted —C₁₋₂ alkyl; and R⁴ is —CH₂CR₃ or —(CH₂)₂CR₃; each R is independently —H or —F; and each of X¹, X², X³, X⁴, X⁵, X⁶, Y¹, Y², Y³, Z¹, Z², Z³, Z⁴, and Z⁵ is independently —H or -D, provided that i) at least two of X¹, X², X³, X⁴, X⁵, X⁶, Y¹, Y², Z¹, Z², Z³, Z⁴, and Z⁵ is independently -D; and ii) when X⁵ is -D, then X⁶ is also -D.

In some embodiments, at least two of X¹, X², X³, and X⁴ is -D. In some instances, at least three of X¹, X², X³, and X⁴ is -D. In other instances, each of X¹, X², X³, and X⁴ is -D.

In some embodiments, Y¹, Y², Y³, Z¹, Z², Z³, Z⁴, and Z⁵ are each —H.

In some embodiments, Y¹, Y², Y³, Z¹, Z², Z³, Z⁴, and Z⁵ are each -D.

In some embodiments, X¹ is -D.

In some embodiments, X⁵ and X⁶ are -D.

In some embodiments, X⁵ and X⁶ are —H.

In some embodiments, the compound of Formula I is a compound of Formula I-g:

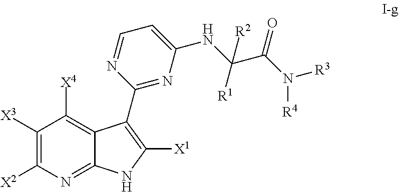

or a pharmaceutically acceptable salt thereof, wherein R¹ is —C(Y¹)(Y²)(Y³); R² is —C(Z¹)(Z²)—C(Z³)(Z⁴)(Z⁵); R³ is —H or unsubstituted —C₁₋₂ alkyl; and R⁴ is —CH₂CR₃ or —(CH₂)₂CR₃; each R is independently —H or —F; and each of X¹, X², X³, X⁴, Y¹, Y², Y³, Z¹, Z², Z³, Z⁴, and Z⁵ is independently —H or -D, provided that at least two of X¹, X², X³, X⁴, Y¹, Y², Y³, Z¹, Z², Z³, Z⁴, and Z⁵ is independently -D.

In one embodiment, X¹, Y¹, Y², Y³, Z¹, Z², Z³, Z⁴, and Z⁵ are independently -D.

In one embodiment, Y¹, Y², Y³, Z¹, Z², Z³, Z⁴, and Z⁵ are independently -D.

In one embodiment, X¹, and X³ are independently -D.

In one embodiment, X¹, X², X³, Y¹, Y², Y³, Z¹, Z², Z³, Z⁴, and Z⁵ are independently -D.

In one embodiment, X¹, X², X³, X⁴, Y¹, Y², Y³, Z¹, Z², Z³, Z⁴, and Z⁵ are independently -D.

In one embodiment, X¹, X², X³, and X⁴ are independently -D.

In some embodiments, the compound of Formula I is a compound of Formula I-h:

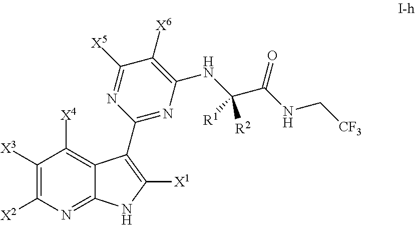

or a pharmaceutically acceptable salt thereof, wherein R¹ is —C(Y¹)(Y²)(Y³); R² is —C(Z¹)(Z²)—C(Z³)(Z⁴)(Z⁵); and each of X¹, X², X³, X⁴, X⁵, X⁶, Y¹, Y², Y³, Z¹, Z², Z³, Z⁴, and Z⁵ is independently —H or -D, provided that at least two of X¹, X², X³, ⁴, X⁵, X⁶, Y¹, Y², Y³, Z¹, Z², Z³, Z⁴, and Z⁵ is independently.

In one embodiment, X¹, Y¹, Y², Y³, Z¹, Z², Z³, Z⁴, and Z⁵ are independently -D.

In one embodiment, Y¹, Y², Y³, Z¹, Z², Z³, Z⁴and Z⁵ are independently -D.

In one embodiment, X¹, and X³ are independently -D.

In one embodiment, X¹, X³, Y¹, Y², Y³, Z¹, Z², Z³, Z⁴, and Z⁵ are independently -D.

In one embodiment, X¹, X², X³, X⁴, Y¹, Y², Y³, Z¹, Z², Z³, Z⁴, and Z⁵ are independently -D.

In one embodiment, X¹, X², X³, and X⁴ are independently -D.

In one embodiment, X¹, X⁵, X⁶, Y¹, Y², Y³, Z¹, Z², Z³, Z⁴, and Z⁵ are independently -D.

In one embodiment, X¹, X², X⁴, and X⁵ are independently -D.

Another aspect of the present invention provides a compound of Formula II:

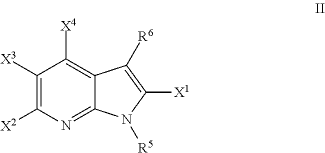

or a pharmaceutically acceptable salt thereof, wherein each of X¹, X², X³, and X⁴ is independently —H or -D; R⁵ is —H or —PG¹, wherein —PG¹ is an amine protecting group; and R⁶ is —H, halo, or —B(OR⁷)₂, wherein each R⁷ is independently —H, —C₁-₄ alkyl, or two —OR⁷ groups taken together with the boron atom to which they are attached form a 5-6 membered heterocycle optionally substituted with 1-4 C₁₋₃ alkyl groups.

In some embodiments, R⁵ is —PG¹, and —PG¹ is —SO₂phenyl. In other embodiments, —PG¹ is a tosyl or Boc protecting group.

In some embodiments, at least one of X¹, X², X³, and X⁴ is -D. For example, at least two of X¹, X², X³, and X⁴ is D. In some instances, at least three of X¹, X², X³, and X⁴ is -D. In other instances, each of X¹, X², X³, and X⁴ is -D.

In some embodiments, X¹ is -D.

Another aspect of the present invention provides a compound of Formula III:

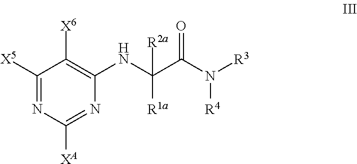

or a pharmaceutically acceptable salt thereof, wherein X^{A} is a leaving group; each of X⁵ and X⁶ is independently —H or -D; R^{1a} is C₁₋₄ alkyl having 1 to 3 hydrogen atoms replaced with deuterium atoms; R^{2a} is C₂₋₄ alkyl having 1 to 7 hydrogen atoms replaced with deuterium atoms; R³ is —H or unsubstituted C₁₋₂ alkyl; and R⁴ is —CH₂CR₃ or —(CH₂)₂CR₃ wherein each R is independently —H or —F.

In some embodiments, X⁵ and X⁶ are each -D.

In other embodiments, X⁵ and X⁶ are each —H.

In some embodiments, X^{A} is halo. For example, X^{A} is —Cl or —Br.

In some embodiments, R^{1a} is methyl having 1 to 3 hydrogen atoms replaced with deuterium atoms. For example, R^{1a} is methyl having 3 hydrogen atoms replaced with deuterium atoms.

In some embodiments, R^{1a} is ethyl having 1 to 5 hydrogen atoms replaced with deuterium atoms. For example, R^{1a} is ethyl having 5 hydrogen atoms replaced with deuterium atoms.

In some embodiments, R^{2a} is ethyl having 1 to 5 hydrogen atoms replaced with deuterium atoms. For example, R^{2a} is ethyl having 5 hydrogen atoms replaced with deuterium atoms.

In some embodiments, R^{2a} is propyl having 1 to 7 hydrogen atoms replaced with deuterium atoms. For example, R^{2a} is propyl having 7 hydrogen atoms replaced with deuterium atoms.

In some embodiments, R³ is —H or methyl. For example, R³ is —H.

In some embodiments, R⁴ is —CH₂CF₃.

In another aspect, the present invention provides Compound 1-a:

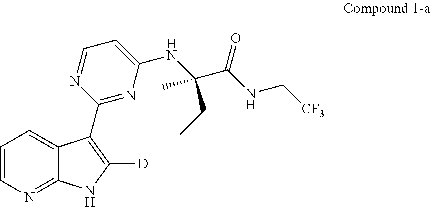 [SMILES: [2H]c1[nH]c2ncccc2c1-c1nccc(N[C@](C)(CC)C(=O)NCC(F)(F)F)n1]

or a pharmaceutically acceptable salt thereof.

Another aspect of the present invention provides a compound of Formula II-a:

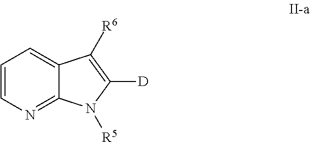

or a pharmaceutically acceptable salt thereof, wherein R⁵ is —H or —PG¹, wherein PG¹ is an amine protecting group; and R⁶ is —H, halo, or —B(OR⁷)₂, wherein each R⁷ is independently —H, —C₁₋₄ alkyl, or two —OR⁷ groups taken together with the boron atom to which they are attached form a 5-6 membered heterocycle optionally substituted with 1-4 —C₁₋₃ alkyl groups.

In some embodiments, R⁵ is —PG¹, and —PG¹ is —SO₂-phenyl or Boc, wherein the phenyl is optionally substituted with alkyl (e.g., methyl). In some instances PG¹ is —SO₂-phenyl, wherein the phenyl is unsubstituted. In other instances, PG¹ is a tosyl protecting group. In another embodiment, PG¹ is a Boc protecting group.

In some embodiments, R⁶ is halo or —B(OR⁷)₂. In a further embodiment, R⁶ is halo. In still a further embodiment, R⁶ is —Cl or —Br. In one embodiment, R⁶ is Br.

In another embodiment, R⁶ is —B(OR⁷)₂, and each R⁷ is hydrogen.

Another aspect of the present invention provides a compound of Formula III-a:

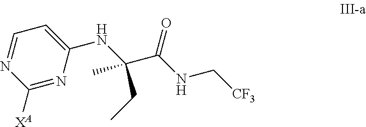

or a pharmaceutically acceptable salt thereof, wherein each X^{A} is a leaving group.

In some embodiments, X^{A} is halo. For example, X^{A} is —Cl or —Br.

### III. SYNTHETIC PROCESSES

Another aspect of the present invention provides a process for preparing a compound of Formula I:

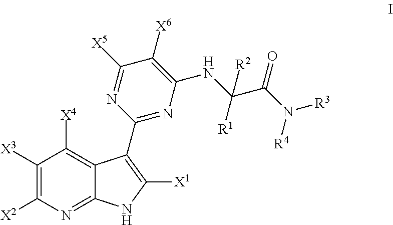

or a pharmaceutically acceptable salt thereof, wherein each of X¹, X², X³, X⁴, X⁵, and X⁶ is independently —H or -D; R¹ is C₁₋₄ alkyl having 0 to 3 hydrogen atoms replaced with deuterium atoms; R² is —C₂₋₄ alkyl having 0 to 7 hydrogen atoms replaced with deuterium atoms; R³ is —H or unsubstituted —C₁₋₂ alkyl; and R⁴ is —CH₂CR₃ or —(CH₂)₂CR₃ wherein each R is independently —H or —F; provided that i) at least one of X¹, X², X³, X⁵, and X⁶ is -D, or at least one of R¹ and R² has at least 1 hydrogen atom that is replaced with a deuterium atom; and ii) when X⁵ is then X⁶ is -D or X² is -H, comprising the steps of:

a) reacting a compound of Formula 1, wherein each R⁷ is independently —H, —C₁₋₄ alkyl, or two —OR⁷ groups taken together with the boron atom to which they are attached form a 5-6 membered heterocycle optionally substituted with 1-4 C₁₋₃ alkyl groups, and PG¹ is an amine protecting group, with a compound of Formula 2, wherein X^{A} is a leaving group,

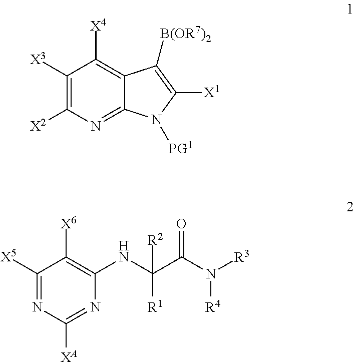

in the presence of a base and a palladium catalyst to generate a compound of Formula 3, and

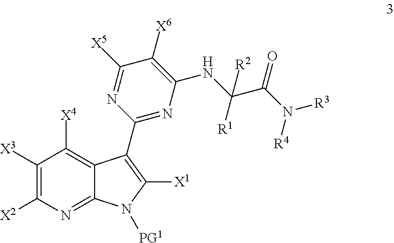

b) deprotecting the compound of Formula 3 to generate the compound of Formula I.

In still another aspect, the present invention provides a process for preparing a compound of Formula I:

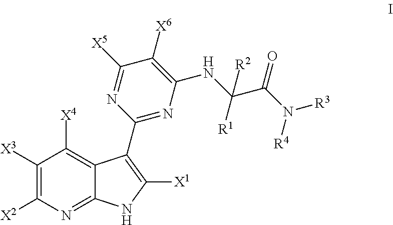

or a pharmaceutically acceptable salt thereof, wherein each of X¹, X², X³, X⁴, X⁵, and X⁶ is independently —H or -D; R′ is C₁₋₄ alkyl having 0 to 3 hydrogen atoms replaced with deuterium atoms; R² is C₂₋₄ alkyl having 0 to 7 hydrogen atoms replaced with deuterium atoms; R³ is —H or unsubstituted —C₁₋₂ alkyl; and R⁴ is —CH₂CR₃ or —(CH₂)₂CR₃ wherein each R is independently —H or —F; provided that i) the compound of Formula I has at least two deuteriums; and ii) when X⁵ is -D, then X⁶ is -D or X² is —H, comprising the steps of:

a) reacting a compound of Formula 1, wherein each R⁷ is independently —H, —C₁₋₄ alkyl, or two —OR⁷ groups taken together with the boron atom to which they are attached form a 5-6 membered heterocycle optionally substituted with 1-4 C₁₋₃ alkyl groups, and PG¹ is an amine protecting group, with a compound of Formula 2, wherein X^{A} is a leaving group,

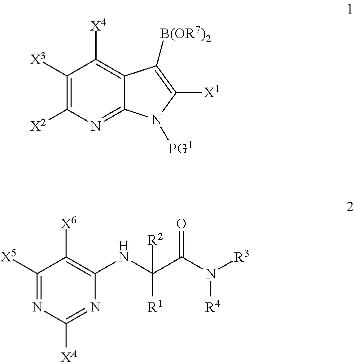

in the presence of a base and a palladium catalyst to generate a compound of Formula 3, and

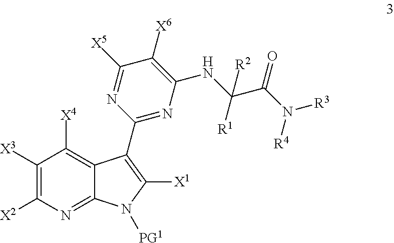

b) deprotecting the compound of Formula 3 to generate the compound of Formula I.

In some embodiments, X^{A} is halo. For example, X^{A} is —Cl or —Br.

Some embodiments further comprise step c) reacting a compound of Formula 4:

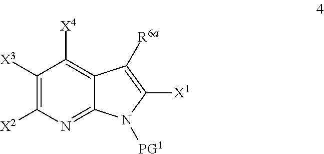

wherein R^{6a} is a leaving group, with a borylating agent to generate the compound of Formula 1.

In some embodiments, the borylating agent comprises bis-pinacol borane. In other embodiments, the borylating agent comprises 2-isopropoxy-4,4,5,5-tetramethyl-1,3,2-dioxaborolane.

Some embodiments further comprise step d) reacting a compound of Formula 5:

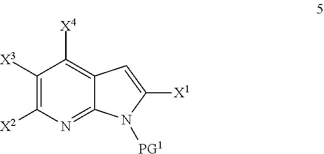

with R^{6a}—X^{B}, wherein X^{B} is halo, in the presence of an organic solvent to generate the compound of Formula 4.

In some embodiments, R^{6a}—X^{B} is Br₂.

Some embodiments further comprise steps e) protecting the compound of Formula 6:

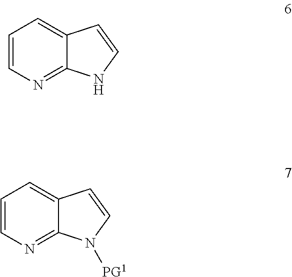

with amine protecting group PG¹, to generate the compound of Formula 7 and reacting the compound of Formula 7 with a deuterating agent to generate the compound of Formula 5.

In some embodiments, PG¹ is —SO₂-phenyl, wherein the phenyl is optionally substituted with alkyl. In some instances PG¹ is —SO₂-phenyl, wherein the phenyl is unsubstituted. In other embodiments, PG¹ is a tosyl or Boc protecting group.

Another aspect of the present invention provides a process for preparing a compound of Formula Ib-1:

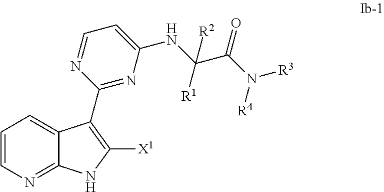

or a pharmaceutically acceptable salt thereof, wherein X¹, is -D; R¹ is C₁₋₄ alkyl having 0 to 3 hydrogen atoms replaced with deuterium atoms; R² is C₂₋₄ alkyl having 0 to 7 hydrogen atoms replaced with deuterium atoms; R³ is —H or unsubstituted —C₁₋₂ alkyl; and R⁴ is —CH₂CR₃ or —(CH₂)₂CR₃ wherein each R is independently —H or —F; comprising the steps of:

a-1) reacting a compound of Formula 1-1, wherein each R⁷ is independently —H, —C₁₋₄ alkyl, or two —OR⁷ groups taken together with the boron atom to which they are attached form a 5-6 membered heterocycle optionally substituted with 1-4 C₁₋₃ alkyl groups, and PG¹ is an amine protecting group, with a compound of Formula 2-1, wherein X^{A} is a leaving group,

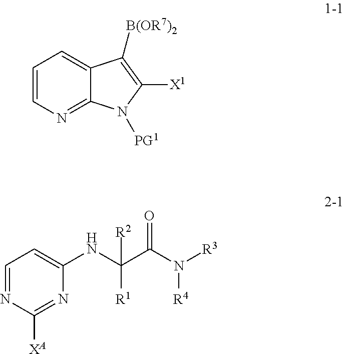

in the presence of a base and a palladium catalyst to generate a compound of Formula 3-1, and

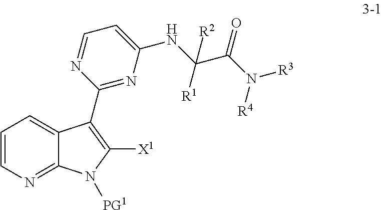

b-1) deprotecting the compound of Formula 3-1 to generate the compound of Formula 5-1.

In some embodiments, X^{A} is halo. For example, X^{A} is —Cl or —Br.

Some embodiments further comprise step c-1) reacting a compound of Formula 4-1:

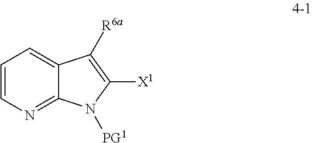

wherein R^{6a} is a leaving group, with a borylating agent to generate the compound of Formula 1-1.

In some embodiments, the borylating agent comprises bis-pinacol borane. In other embodiments, the borylating agent comprises 2-isopropoxy-4,4,5,5-tetramethyl-1,3,2-dioxaboro lane.

Some embodiments further comprise step d-1) reacting a compound of Formula 5-1:

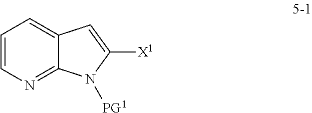

with R^{6a}—X^{B}, wherein X^{B} is halo, in the presence of an organic solvent to generate the compound of Formula 4-1.

In some embodiments, R^{6a}—X^{B} is Br₂.

Some embodiments further comprise steps e) protecting the compound of Formula 6:

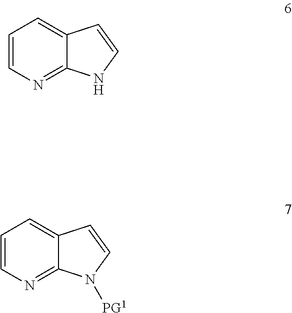

with amine protecting group PG¹, to generate the compound of Formula 7 and reacting the compound of Formula 7 with a deuterating agent to generate the compound of Formula 5-1.

In some embodiments, PG¹ is —SO₂-phenyl, wherein the phenyl is optionally substituted with alkyl. In other examples, the phenyl is unsubstituted. In other embodiments, —PG¹ is a tosyl or Boc protecting group.

Another aspect of the present invention provides a process for preparing Compound 1-a:

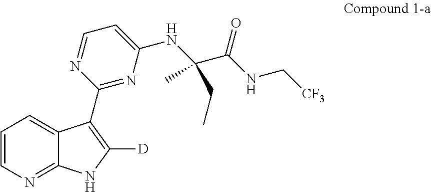 [SMILES: [2H]c1[nH]c2ncccc2c1-c1nccc(N[C@](C)(CC)C(=O)NCC(F)(F)F)n1]

or a pharmaceutically acceptable salt thereof, comprising the steps of:

a-2) reacting a compound of Formula 1-1a, wherein each R⁷ is independently —H, —C₁₋₄ alkyl, or two —OR⁷ groups taken together with the boron atom to which they are attached form a 5-6 membered heterocycle optionally substituted with 1-4 C₁₋₃ alkyl groups, and PG¹ is an amine protecting group, with a compound of Formula III-a, wherein X^{A} is a leaving group,

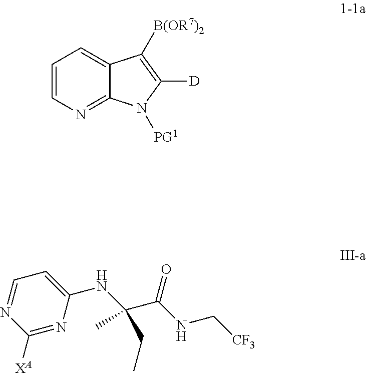

in the presence of a base and a palladium catalyst to generate a compound of Formula IV, and

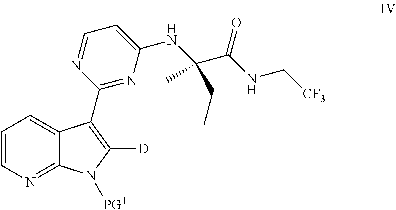

b-2) deprotecting the compound of Formula IV to generate Compound 1-a.

In some embodiments, X^{A} is halo. For example, X^{A} is —Cl or —Br.

Some embodiments further comprise step c-2) reacting a compound of Formula 4-2:

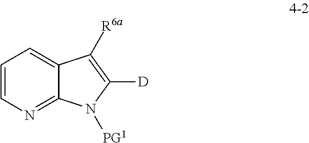

wherein R^{6a} is a leaving group, with a borylating agent to generate the compound of Formula 1-1a.

In some embodiments, the borylating agent comprises bis-pinacol borane. In some embodiments, the borylaying agent comprises 2-isopropoxy-4,4,5,5-tetramethyl-1,3,2-dioxaborolane.

Some embodiments further comprise step d-2) reacting a compound of Formula VI:

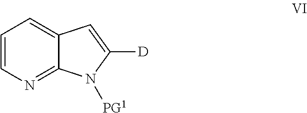

with R^{6a}—X^{B}, wherein X^{B} is halo, in the presence of an organic solvent to generate the compound of Formula 4-2.

In some embodiments, R^{6a}—X^{B} is Br₂.

Some embodiments further comprise steps e) protecting the compound of Formula 6:

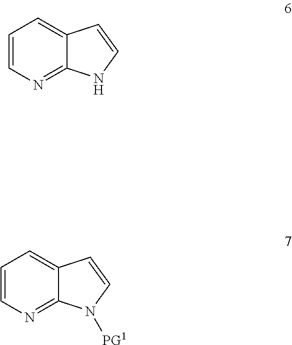

with an amine protecting group PG¹, to generate the compound of Formula 7 and reacting the compound of Formula 7 with a deuterating agent to generate the compound of Formula VI.

In some embodiments, PG¹ is —SO₂-phenyl or Boc, wherein the phenyl is optionally substituted with alkyl. In some instances PG¹ is —SO₂-phenyl, wherein the phenyl is unsubstituted. In one embodiment, PG¹ is a tosyl protecting group. In another embodiment, —PG¹ is a Boc protecting group.

A. Steps a), a-1), or a-2)

In some embodiments, palladium catalyst of step a), step a-1), or step a-2) comprises palladium(II)acetate, tetrakis(triphenylphosphine)palladium(O), tris(dibenzylideneacetone)dipalladium(O), or any combination thereof. In some embodiments, the palladium-based catalyst comprises tetrakis(triphenylphosphine)palladium(0).

In some embodiments, the palladium catalyst is formed in situ.

In some embodiments, the base of step a), step a-1), or step a-2) is an inorganic base. Examples of inorganic bases include tripotassium phosphate, dipotassium hydrogen phosphate, dipotassium carbonate, disodium carbonate, trisodium phosphate, or disodium hydrogen phosphate. In some embodiments, the inorganic base is tripotassium phosphate, dipotassium hydrogen phosphate, trisodium phosphate, or disodium hydrogen phosphate. In other embodiments, the inorganic base is disodium carbonate. Other examples of inorganic bases include alkali metal hydroxides such as NaOH, KOH, or any combination thereof.

In some embodiments, the reaction of step a), step a-1), or step a-2) is performed in the presence of an aprotic solvent. For example, the aprotic solvent of step a), step a-1), or step a-2) comprises acetonitrile, toluene, N,N-dimethylformamide, N,N-dimethylacetamide, acetone, methyl tent-butyl ether, or any combination thereof. In other examples, the aprotic solvent is N,N-dimethylacetamide.

In some embodiments, the reaction of step a), step a-1), or step a-2) is performed at a temperature between about 60° C. and about 120° C. For example, the reaction of step a), step a-1), or step a-2) is performed at a temperature between about 70° C. and about 110° C. In other embodiments, the reaction of step a), step a-1), or step a-2) is performed at a temperature between about 80° C. and about 100° C.

In some embodiments, step a), step a-1), or step a-2) is performed with agitation. For example, the reaction is performed in a vessel containing a stir bar that agitates the reaction mixture.

B. Steps b), b-1), or b-3)

In some embodiments, the deprotection of the compound of Formula 3, Formula 3-1, or Formula IV is performed in the presence of a base. In some examples, the base comprises an inorganic base such as an alkali metal hydroxide. Examples of alkali metal hydroxides include LiOH, NaOH, KOH, or any combination thereof. In other embodiments, step b), step b-1), or step b-2) comprises deprotecting the compound of Formula 3, Formula 3-1 or Formula IV in the presence of LiOH.

In some embodiments, the alkali-metal hydroxide base has a concentration of about 1N to about 6N. In other embodiments, the alkali-metal hydroxide base has a concentration of about 2N.

In some embodiments, the deprotection reaction in step b), step b-1), or step b-2) is performed at a temperature between about 60° C. and about 120° C. For example the deprotection reaction in step b), step b-1), or step b-2) is performed at a temperature between about 70° C. and about 110° C. In other examples, the deprotection reaction in step b), step b-1), or step b-2) is performed at a temperature between about 80° C. and about 100° C.

C. Step c), c-1, or c-2)

In step c), step c-1), or step c-2), the compound of Formula 4, Formula 4-1, or Formula 4-2 reacts with a borylating agent to generate the compound of Formula Ha, Formula 1-1, or Formula 1-1a. In some embodiments, the borylating agent comprises bis-pinacol borane. In other embodiments, the borylating agent comprises 2-isopropoxy-4,4,5,5-tetramethyl-1,3,2-dioxaborolane.

In some embodiments, the reaction of step c), step c-1), or step c-2) is performed in the presence of an organic solvent. For example, the reaction of step c), step c-1), or step c-2) is performed in the presence of 1,2-dimethoxyethane, THF, methyl-THF, 1,4-dioxane or any combination thereof.

In some embodiments, the reaction of step c), step c-1), or step c-2) is performed in the presence of a transition metal catalyst. In some examples, the transition metal catalyst is a palladium catalyst. For instance, the palladium metal catalyst comprises Pd(dppf)Cl₂.

D. Additional Steps

In some embodiments, the reaction of step d) or step d-1) is performed in the presence of a polar organic solvent. Examples of polar organic solvents useful for performing the reaction of step d) or step d-1) include dichloromethane, chloroform, or any combination thereof.

In some embodiments, the reaction of step e) is performed in the presence of an organic solvent. Organic solvents useful for step e) include ether(s), THF, methyl-THF, DME, or any combination thereof.

In some embodiments, the deuterating agent of step f) is D₂O, CD₃OD, or any combination thereof. And, in some embodiments, step f) is repeated one or more times.

### IV. PROCESS AND INTERMEDIATES

The following definitions describe terms and abbreviations used herein:
Ac acetyl
Bu butyl
Et ethyl
Ph phenyl
Me methyl
THF tetrahydrofuran
DCM dichloromethane
CH₂Cl₂ dichloromethane
EtOAc ethyl acetate
CH₃CN acetonitrile
EtOH ethanol
MeOH methanol
MTBE methyl tent-butyl ether
DMF N,N-dimethylformamide
DMA N,N-dimethylacetamide
DME dimethylether
DMSO dimethyl sulfoxide
HOAc acetic acid
TFA trifluoroacetic acid
Et₃N triethylamine
DIPEA diisopropylethylamine
DIEA diisopropylethylamine
K₂CO₃ dipotassium carbonate
Na₂CO₃ disodium carbonate
NaOH sodium hydroxide
K₃PO₄ tripotassium phosphate
HPLC high performance liquid chromatography
Hr or h hours
atm atmospheres
rt or RT room temperature
HCl hydrochloric acid
HBr hydrobromic acid
H₂O water
NaOAc sodium acetate
H₂SO₄ sulfuric acid
N₂ nitrogen gas
H₂ hydrogen gas
Br₂ bromine
n-BuLi n-butyl lithium
Pd(OAc)₂ palladium(II)acetate
PPh₃ triphenylphosphine
rpm revolutions per minute
Equiv. equivalents
Ts tosyl
IPA isopropyl alcohol

As used herein, other abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997, herein incorporated in its entirety by reference.

In one embodiment, the invention provides a process and intermediates for preparing a compound of Formula I as outlined in Scheme I.

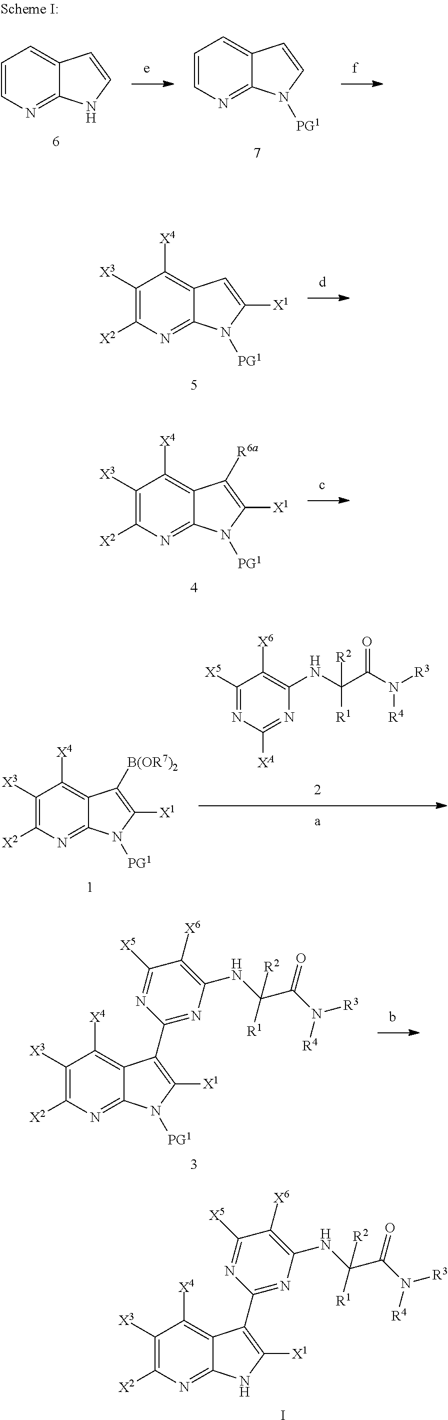

In Scheme I, the starting material, i.e., the compound of Formula 6, is reacted with PG¹—X^{C}, wherein X^{C} is halo, (e.g., benzenesulfonyl chloride) to generate the protected compound of Formula 7. The compound of Formula 7 is deuterated using a deuterating agent (e.g., D₂O) to generate the deuterated compound of Formula 5. The deuterated compound of Formula 5 is reacted with R^{6a}—X^{B} to generate the compound of Formula 4, which is borylated to generate the compound of Formula 1. The compound of Formula 1 is coupled with the compound of Formula 2 via a palladium catalyzed cross coupling reaction to generate the compound of Formula 3, which undergoes deprotection to generate the compound of Formula I.

In one embodiment, the invention provides a process and intermediates for preparing a compound of Formula I as outlined in Scheme II.

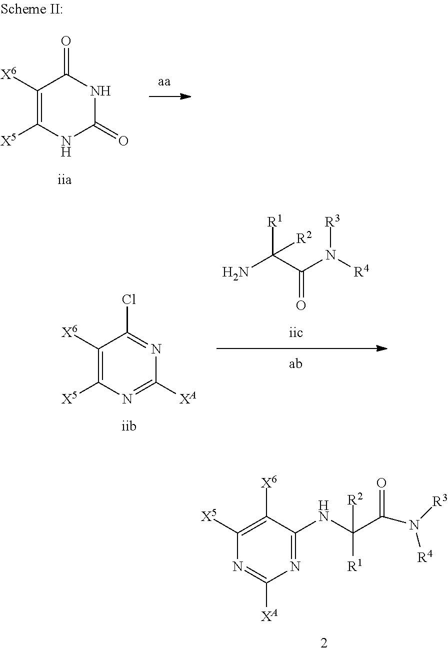

In Scheme II, the compound of Formula iia, wherein X⁵ and X⁶ are defined above, is reduced to generate the protected compound of Formula iib, wherein X^{A} is defined above. The compound of Formula iib is reacted with the compound of Formula iic under coupling conditions, to generate the compound of Formula 2. Note that each of X^{A}, X⁵, X⁶, R¹, R², R³, and R⁴ are as defined above.

### V. EXAMPLES

The following preparative examples are set forth in order that this invention is more fully understood. These examples are for the purpose of illustration only and are not to be construed as limiting the scope of the invention in any way.

### Example 1A

### 1-(phenylsulfonyl)-1H-pyrrolo[2,3-b]pyridine

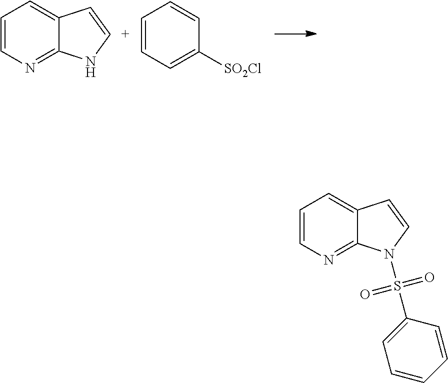 [SMILES: [C+]1=Cc2cccnc2N1 ; O=S(=O)(Cl)c1ccccc1 ; O=S(=O)(c1ccccc1)n1ccc2cccnc21]

In a flask containing 1H-pyrrolo[2,3-b]pyridine (5.03 g, 42.58 mmol) in THF (75 mL) was added at r.t., NaH (4.256 g, 106.4 mmol). After 30 min of stirring, benzenesulfonyl chloride (9.400 g, 6.792 mL, 53.22 mmol) was added. The mixture was stirred at r.t. for 2 hrs. The solvent was evaporated, water added, and the solution extracted with ethyl acetate, washed with brine, and dried over Na₂SO₄. The solvent was filtered and evaporated. The crude product was purified by Chrom./Silica (EtOAc 10-100%/Hex.) to yield 1-(benzenesulfonyppyrrolo[2,3-b]pyridine (8.6 g, 33.30 mmol, 78.18%) ESI-MS m/z calc. 258.0463, found 259.1 (M+1); Retention time: 1.0 min.

### Example 1B

### 1-tosyl-1H-pyrrolo[2,3-b]pyridine

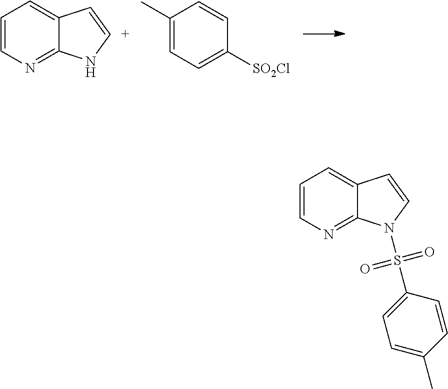 [SMILES: [C+]1=Cc2cccnc2N1 ; Cc1ccc(S(=O)(=O)Cl)cc1 ; Cc1ccc(S(=O)(=O)n2ccc3cccnc32)cc1]

A flask was charged with 1H-pyrrolo[2,3-b]pyridine (350.0 g, 2.963 mol) and toluene (2800 mL), followed by 4-toluenesulfonyl chloride (626.9 g, 3.288 mol) and TBAB (9.55 g, 0.0296 mol) in toluene (2800 mL). An aqueous solution of 25% NaOH (1185.2 g) was added dropwise into the mixture while controlling the temperature between 20-30° C. The mixture was stirred at 20-25° C. overnight. Water (700 ml) and THF (1750 ml) were added to the mixture and aqueous phase was extracted with THF (2×1750 ml). The organic phase was washed with brine (2×1750 mL) and dried over Mg₂SO₄. The organic phase was then concentrated to 700-850 mL and filtered. The cake was then washed with n-heptane (3×350 ml). After drying, 330 g of 1-tosyl-1H-pyrrolo[2,3-b]pyridine was obtained.

### Example 2A

### 2-deuterio-1-(phenylsulfonyl)-1H-pyrrolo [2,3-b]pyridine

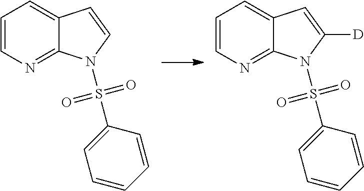 [SMILES: O=S(=O)(c1ccccc1)n1ccc2cccnc21 ; [2H]c1cc2cccnc2n1S(=O)(=O)c1ccccc1]

In a flask containing 1-(benzenesulfonyl)pyrrolo[2,3-b]pyridine (7.95 g, 30.78 mmol) in dry THF (250 mL) under N₂ and at −78° C. was added slowly BuLi (26.77 mL of 2.3 M, 61.56 mmol). After -1 eq. was added, a solid precipitated from the mixture. After 2 hrs of stirring at −78° C., D₂O (18.49 g, 16.66 mL, 923.4 mmol) was added at −78° C. The solution was stirred at −78° C. for another hour. The solution was concentrated, water (200 mL) was added, and the aqueous layer was extracted with EtOAc (2x200 mL). The extract was washed with brine and dried over Na₂SO₄ to yield 2-deuterio-1-(phenylsulfonyl)-1H-pyrrolo[2,3-b]pyridine (7.8 g, 29.78 mmol).

¹H NMR (300 MHz, Methanol-d4) δ 8.32 (dd, J=4.8, 1.6 Hz, 1H), 8.16-8.08 (m, 2H), 7.97 (dd, J=7.9, 1.6 Hz, 1H), 7.67-7.59 (m, 1H), 7.58-7.49 (m, 2H), 7.25 (dd, J=7.9, 4.8 Hz, 1H), 6.73 (s, 1H). ESI-MS m/z calc. 259.05258, found 260.14 (M+1); Retention time: 1.0 min.

### Example 2B

### 2-deuterio-1-(tosyl)-1H-pyrrolo [2,3-b]pyridine

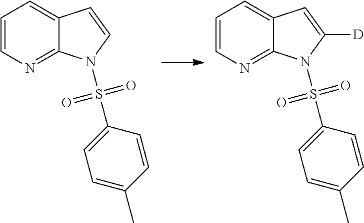 [SMILES: Cc1ccc(S(=O)(=O)n2ccc3cccnc32)cc1 ; [2H]c1cc2cccnc2n1S(=O)(=O)c1ccc(C)cc1]

A solution of 1-tosyl-1H-pyrrolo[2,3-b]pyridine (165.0 g, 0.6059 mol) in dry THF (2975 mL) under N₂ was cooled to -78° C. A solution of BuLi (484 mL of 2.5 M, 1.2118 mol) was added slowly the flask maintaining the temperature <−70° C. After 2 hours of stirring at −78° C., D₂O (182.0 g, 9.0885 mol) was added at −78° C. This mixture was then warmed to 20-25° C. while stirring overnight. A 10% aqueous NaCl solution (825 mL) was then added. After stirring for 30 minutes, the phases were separated. The aqueous phase was extracted with MTBE (2×825 mL). The combined organic phase was washed with brine ° C. (2×825 mL) and dried over Mg₂SO₄. The organic phase was then concentrated to obtain 2-deuterio-1-tosyl-1H-pyrrolo [2,3-b]pyridine (>99% deuteratium incorporation by HNMR).

### Example 3A

### 3-bromo-2-deuterio-1-(phenylsulfonyl)-1H-pyrrolo[2,3-b]pyridine

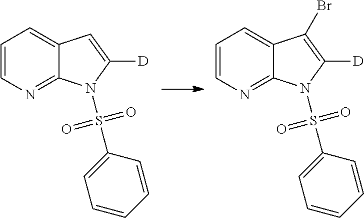 [SMILES: [2H]c1cc2cccnc2n1S(=O)(=O)c1ccccc1 ; [2H]c1c(Br)c2cccnc2n1S(=O)(=O)c1ccccc1]

In a flask containing 2-deuterio-1-(phenylsulfonyl)-1H-pyrrolo[2,3-b]pyridine (7.8 g, 30.08 mmol) in DCM (300 mL) was added dropwise (over 30 min) a solution of bromine (5.288 g, 1.705 mL, 33.09 mmol) in DCM (100 mL). The solution was stirred at r.t. for another 2 hrs. The reaction was quenched with a solution of NaHSO₃, the organic phase was then washed with NaHCO₃ (sat), brine, and dried over MgSO₄. The product was purified by chromatography on ISCO C18 150 g (TFA buffer) to yield 3-bromo-2-deuterio-1-(phenylsulfonyl)-1H-pyrrolo[2,3-b]pyridine (3.75 g, 11.09 mmol, 36.87%). ESI-MS m/z calc. 336.96307, found 338.0 (M+1); Retention time: 1.15 min. Deuterium content analysis by LCMS=D₁˜98%.

### Example 3B

### 3-bromo-2-deuterio-1-(tosyl)-1H-pyrrolo[2.3-b]pyridine

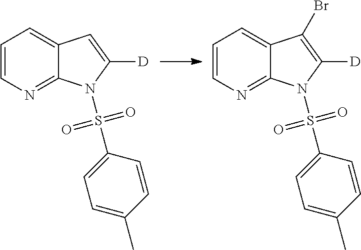 [SMILES: [2H]c1cc2cccnc2n1S(=O)(=O)c1ccc(C)cc1 ; [2H]c1c(Br)c2cccnc2n1S(=O)(=O)c1ccc(C)cc1]

A solution of 2-deuterio-l-(tosyl)-1H-pyrrolo[2,3-b]pyridine (300.0 g, 1.098 mol) in DMF (2400 mL) was cooled to 0-5° C. A solution of Br₂ (192.9 g, 1.207 mol) in DMF (600 mL) was added slowly to the reactor. The reaction mixture was then stirred at 0-5° C. for 4 hours. After confirming reaction completion, a 10% aqueous NaHSO₃ solution was added to the reaction mixture while controlling the temperature to <20° C., followed by the addition of water (4 L). The mixture was then stirred for 0.5 h. The resulting solids were filtered and the cake was washed with water (3 x 600 mL) followed by n-heptane (2 x 600 mL). The yellow solids were then dried to obtain 3-bromo-2-deuterio-1-(tosyl)-1H-pyrrolo[2,3-b]pyridine (266.2 g, >99% deuterium incorporation by HNMR).

### Example 4a

### (2-deuterio-1-(phenylsulfonyl)-1H-pyrrolo[2,3-b]pyridin-3-yl)boronic acid

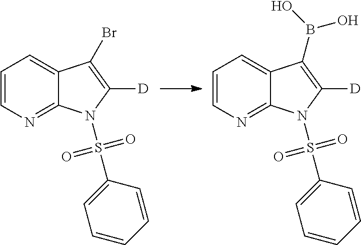 [SMILES: [2H]c1c(Br)c2cccnc2n1S(=O)(=O)c1ccccc1 ; [2H]c1c(B(O)O)c2cccnc2n1S(=O)(=O)c1ccccc1]

In a 250 mL round-bottomed flask equipped with a spin bar and reflux condenser, the 3-bromo-2-deuterio-1-(phenylsulfonyl)-1H-pyrrolo[2,3-b]pyridine (3.75 g, 11.09 mmol), KOAc (3.265 g, 33.27 mmol) and bis-pinacol borane (4.505 g, 17.74 mmol) were added. 1,2-dimethoxyethane (100 mL) was added and the mixture degassed for several minutes. Pd(dppf)Cl₂ (634.0 mg, 0.7763 mmol) was added and the reaction mixture was heated at 90° C. overnight. The reaction mixture was concentrated to reduced volume then filtered through florisil and eluted with DCM. The solvent was evaporated and the residue triturated with ether and hexane followed by several hexane washes to yield (2-deuterio-1-(phenylsulfonyl)-1H-pyrrolo[2,3-b]pyridin-3-yl)boronic acid. ESI-MS m/z calc. 303.05954, found 304.0 (M+1); Retention time: 0.87 min. Deuterium content analysis by LCMS=D₁˜98%.

### Example 4b

### (2-deuterio-1-(phenylsulfonyl)-1H-pyrrolo[2,3-b]pyridin-3-yl)boronic acid

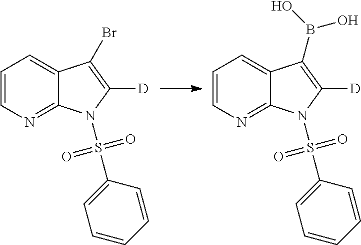 [SMILES: [2H]c1c(Br)c2cccnc2n1S(=O)(=O)c1ccccc1 ; [2H]c1c(B(O)O)c2cccnc2n1S(=O)(=O)c1ccccc1]

In a flask containing 1-(benzenesulfonyl)-3-bromo-2-deuterio-pyrrolo[2,3-b]pyridine (1.53 g, 4.096 mmol) in THF (50 mL) was added triisopropoxyborane (2.311 g, 2.818 mL, 12.29 mmol). The solution was cooled to −78° C. and n-BuLi (1.959 mL of 2.3 M, 4.506 mmol) was added slowly. After 3 hrs, the solution was quenched with D₂O. The mixture was stirred for 1 hr at r.t. The solvent was concentrated to dryness. The product was purified by chromatography on ISCO C18Aq 150 g (TFA buffer) to yield [1-(benzenesulfonyl)-2-deuterio-pyrrolo[2,3-b]pyridin-3-yl]boronic acid.

¹H NMR (300 MHz, Methanol-d4) δ 8.45-8.23 (m, 2H), 8.22-8.05 (m, 2H), 7.72-7.44 (m, 3H), 7.24 (dd, J=7.8, 5.0 Hz, 1H). ESI-MS m/z calc. 303.06, found 304.08 (M+1)⁺; Retention time: 0.72 min. Deuterium content analysis by LCMS=D₁˜98%.

### Example 4C

### 2-deuterio-1-(tosyl)-3-(4,4,5,5-tetramethyl-1,3,2-dioxaborolan-2-yl)-1H-pyrrolo[2,3-b]pyridine

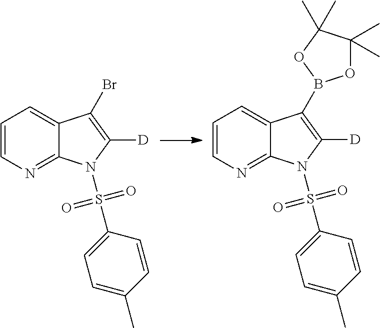 [SMILES: [2H]c1c(Br)c2cccnc2n1S(=O)(=O)c1ccc(C)cc1 ; [2H]c1c(B2OC(C)(C)C(C)(C)O2)c2cccnc2n1S(=O)(=O)c1ccc(C)cc1]

A solution of bis-pinacol borane (265.9 g, 1.0473 mol) and KOAc (205.6 g, 2.0946 mol) in 1,2-dimethoxyethane (3600 mL) was added to a flask containing 3-bromo-2-deuterio-1-(tosyl)-1H-pyrrolo[2,3-b]pyridine (246.0 g, 0.6982 mol). This mixture was degassed for 30 minutes at 20-25° C. Pd(dppf)Cl₂ (35.76 g, 0.04887 mol) was added then the reaction mixture was heated to 85-90° C. After stirring at this temperature for 2-4 hours and confirming reaction completion, activated carbon (12 g) was added. The mixture was stirred for 30 minutes then filtered. The filtrate was concentrated to 480 mL. MTBE (1200 mL) and water (1200 mL) were added. After stirring for 30 minutes, the phases were separated. The aqueous phase was extracted with twice with MTBE (1200 mL then 600 ml). The organic phase was washed with brine (2×1200 mL). The organic phase was dried with MgSO₄ and filtrated through silica gel. The filtrate was concentrated to 480 mL. Isopropyl alcohol (600 mL) was added then the mixture was concentrated to 480 mL. Isopropyl alcohol (600 mL) was added then the mixture was heated at 80-85° C. After stirring for 30 minutes, the mixture was cooled to 5-15° C. The solids were filtered then washed with isopropyl alcohol (240 mL), which was pre-cooled to 5-15° C. followed by n-heptane (2 x 240 mL). The solids were then recrystallized from isopropyl alcohol to obtain 2-deuterio-1-(tosyl)-3-(4,4,5,5-tetramethyl-1,3,2-dioxaborolan-2-yl)-1H-pyrrolo[2,3-b]pyridine as an off white solid (182.0 g, >99% deuterium incorporation by HNMR).

### Example 5

### (2R)-2-[[2-(2-deuterio-1H-pyrrolo[2,3-b]pyridin-3-yl)pyrimidin-4-yl]amino]-2-methyl-N-(2.2.2-trifluoroethyl)butanamide

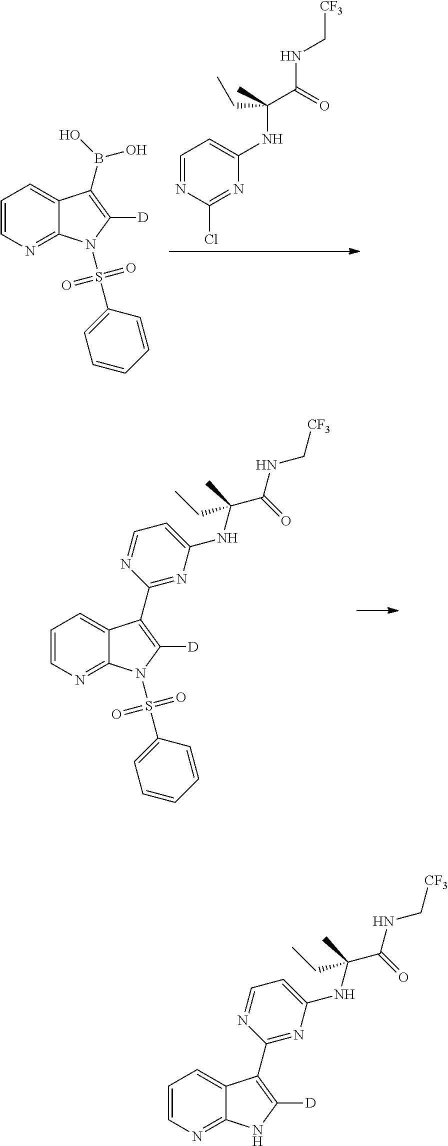 [SMILES: [2H]c1c(B(O)O)c2cccnc2n1S(=O)(=O)c1ccccc1 ; CC[C@@](C)(Nc1ccnc(Cl)n1)C(=O)NCC(F)(F)F ; [2H]c1c(-c2nccc(N[C@](C)(CC)C(=O)NCC(F)(F)F)n2)c2cccnc2n1S(=O)(=O)c1ccccc1 ; [2H]c1[nH]c2ncccc2c1-c1nccc(N[C@](C)(CC)C(=O)NCC(F)(F)F)n1]

In a flask containing [1-(benzenesulfonyl)-2-deuterio-pyrrolo[2,3-b]pyridin-3-yl]boronic acid (120.0 mg, 0.3959 mmol) in DME (4 mL) was added (2R)-2-[(2-chloropyrimidin-4-yl)amino]-2-methyl-N-(2,2,2-trifluoroethyl)butanamide (135.3 mg, 0.4355 mmol) and Na₂CO₃ (594.0 _{[}IL of 2 M, 1.188 mmol). The solution was degassed for several minutes with N₂. Pd(PPh₃)₄ (22.88 mg, 0.01980 mmol) was added and the solution heated to 90° C. for 3 hrs. The solution was filtered over a pad of Florisillcelite and washed with DCM. The solvent was evaporated and the crude product was purified by chromatography on ISCO C18 100 g (TFA buffer) to yield (2R)-24[241-(benzenesulfonyl)-2-deuterio-pyrrolo[2,3-b]pyridin-3-yl]pyrimidin-4-yl]amino]-2-methyl-N-(2,2,2-trifluoroethypbutanamide (148 mg, 0.2774 mmol).

¹H NMR (300 MHz, Methanol-d4) δ 8.67 (d, J=8.0 Hz, 1H), 8.59 (t, J=6.2 Hz, 1H), 8.47 (dd, J=4.7, 1.6 Hz, 1H), 8.26 (dd, J=7.6, 1.8 Hz, 2H), 8.17 (d, J=7.2 Hz, 1H), 7.72 (dd, J=8.6, 6.2 Hz, 1H), 7.61 (t, J=7.5 Hz, 2H), 7.49-7.41 (m, 1H), 6.84 (d, J=7.2 Hz, 1H), 3.75 (q, J=8.3 Hz, 2H), 2.10 (ddd, J=52.4, 14.0, 7.2 Hz, 2H), 1.69 (s, 3H), 0.96 (t, J=7.5 Hz, 3H). ESI-MS m/z calc. 533.15674, found 534.1 (M+1); Retention time: 0.83 min.

Hydrolysis of the PhSO₂ was accomplished with LiOH (2N)/MeOH at 90° C. for 1 hr. The solvent was evaporated, and the product was purified by chromatography on ISCO C18 100 g (TFA buffer). The resulting compound was neutralized using a SPE-CO_{S}H cartridge (eluted with DCM) to yield (2R)-2-[[2-[2-deuterio-pyrrolo[2,3-b]pyridin-3-yl]pyrimidin-4-yl]amino]-2-methyl-N-(2,2,2-trifluoroethyl)butanamide. ¹HNMR and LCMS showed 98% deuterium incorporation at position 2 of the azaindole.

¹H NMR (300 MHz, Methanol-d4) δ 8.82 (dd, J=7.9, 1.6 Hz, 1H), 8.22 (dd, J=4.8, 1.6 Hz, 1H), 8.10 (d, J=6.0 Hz, 1H), 7.22 (dd, J=8.0, 4.8 Hz, 1H), 6.42 (d, J=6.0 Hz, 1H), 3.96-3.61 (m, 2H), 2.22 (dq, J=15.0, 7.6 Hz, 1H), 1.92 (dq, J=13.6, 7.5 Hz, 1H), 1.61 (s, 3H), 0.94 (t, J=7.5 Hz, 3H). ESI-MS m/z calc. 393.1635, found 394.11 (M+1); Retention time: 0.64 min.

### Example 6

### Assessment of Metabolite Profile and Kinetic Isotope Effect of Compound 1-a

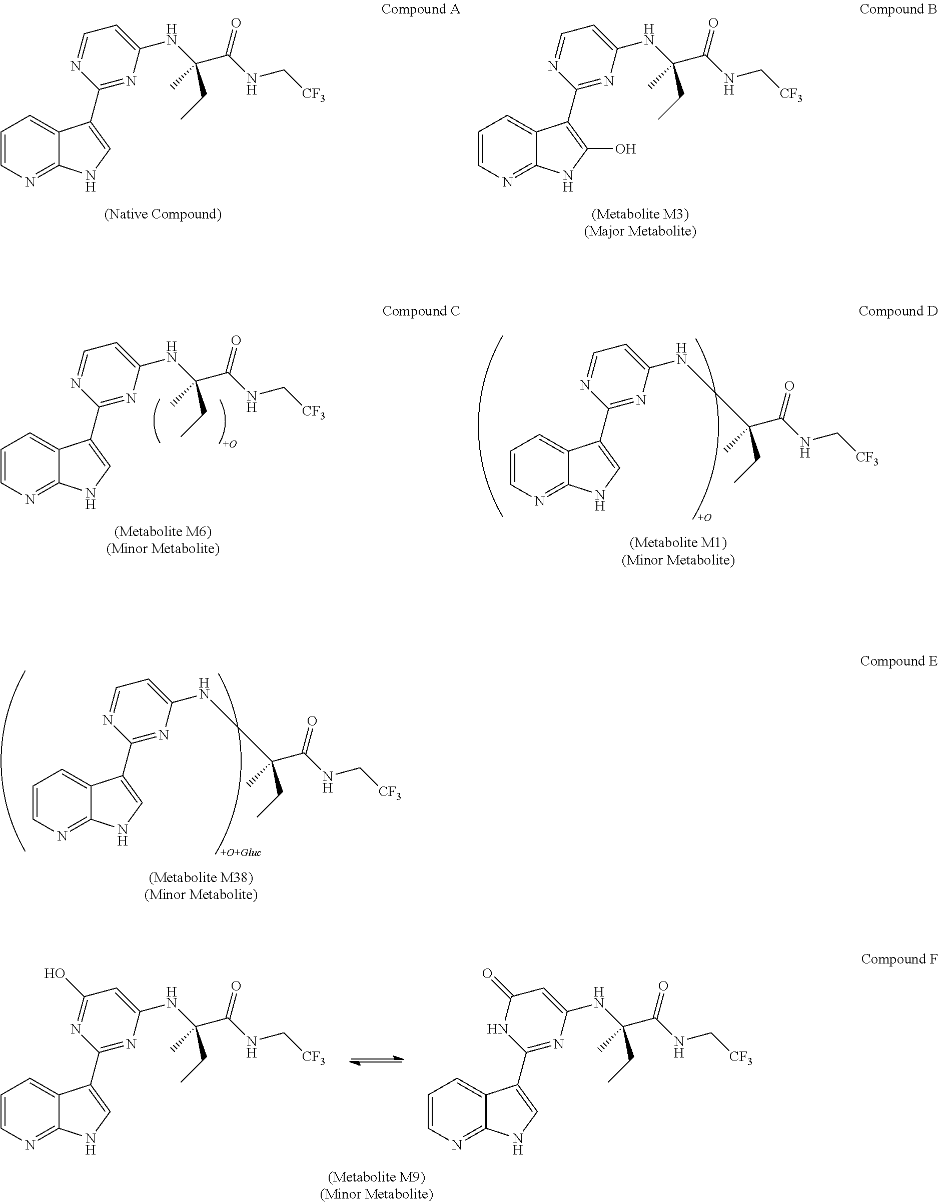

TABLE 1
Compounds Assayed in Example 6.

| 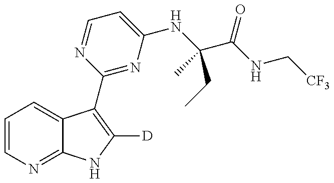 [SMILES: [2H]c1[nH]c2ncccc2c1-c1nccc(N[C@](C)(CC)C(=O)NCC(F)(F)F)n1] | 1-a |
|---|---|
| 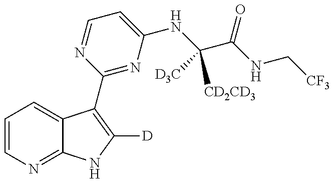 | 2 |
| 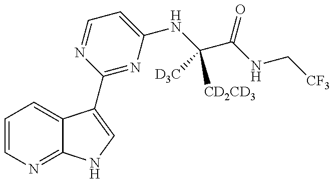 | 3 |
| 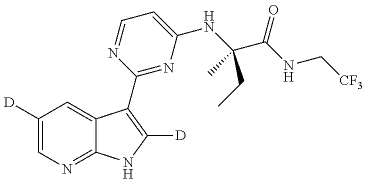 [SMILES: [2H]c1cnc2[nH]c([2H])c(-c3nccc(N[C@](C)(CC)C(=O)NCC(F)(F)F)n3)c2c1] | 4 |
| 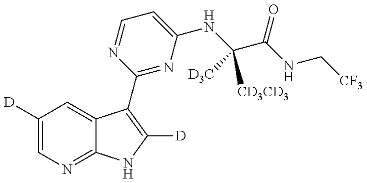 | 5 |
| 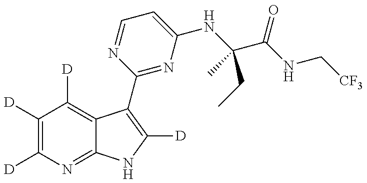 [SMILES: [2H]c1nc2[nH]c([2H])c(-c3nccc(N[C@](C)(CC)C(=O)NCC(F)(F)F)n3)c2c([2H])c1[2H]] | 6 |
| 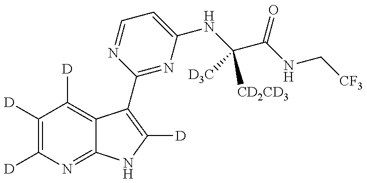 | 7 |
| 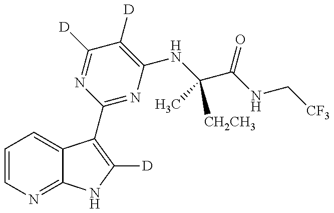 [SMILES: [2H]c1nc(-c2c([2H])[nH]c3ncccc23)nc(N[C@](C)(CC)C(=O)NCC(F)(F)F)c1[2H]] | 8 |
| 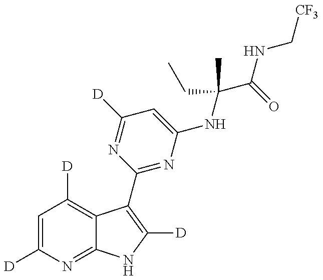 [SMILES: [2H]c1cc(N[C@](C)(CC)C(=O)NCC(F)(F)F)nc(-c2c([2H])[nH]c3nc([2H])cc([2H])c23)n1] | 9 |

Incubation Details

Cryopreserved Human Hepatocytes lot TFF (purchased from Celsis) were used. Hepatocytes were thawed using CHRM and suspended in Williams E media containing cell maintenance supplement package. 1000 μL of a final cell concentration 1 million cells/mL were placed in individual incubation wells (24-well plate set-up). Incubation was conducted at 37° C. and kept in a CO₂/O₂ humidified incubator. 10 μL of compound stock was spiked into cell the matrix to achieve final incubation concentrations of 3 μM and 10 μM. The matrix was swirled prior to the removal of each time-point and 50 μL of sample were removed and added to 200 μL of acetonitrile containing internal standard, IS. Time-points were sampled at 120 minutes using MRM on an ABSciex API5500-QTrap paired with an Agilent 1290 UPLC and a CTC PAL autosampler. A 20-minute gradient method using a HALO C18 2.1×50 mm 2.7 μm column made by Advanced Materials Technology was used for the analysis. /

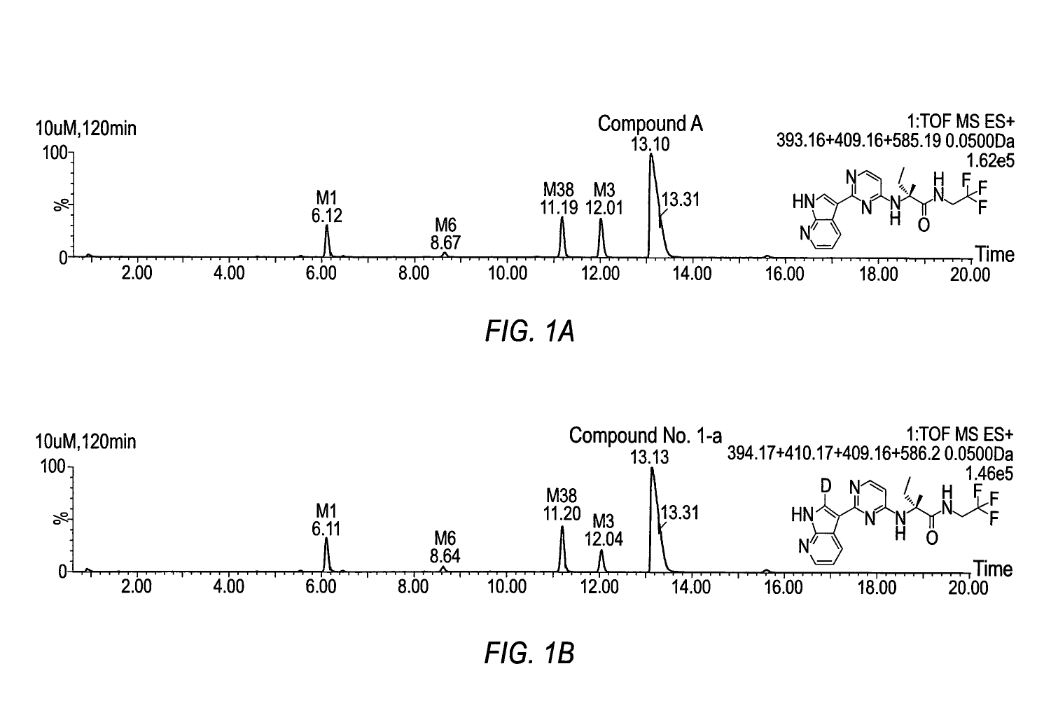

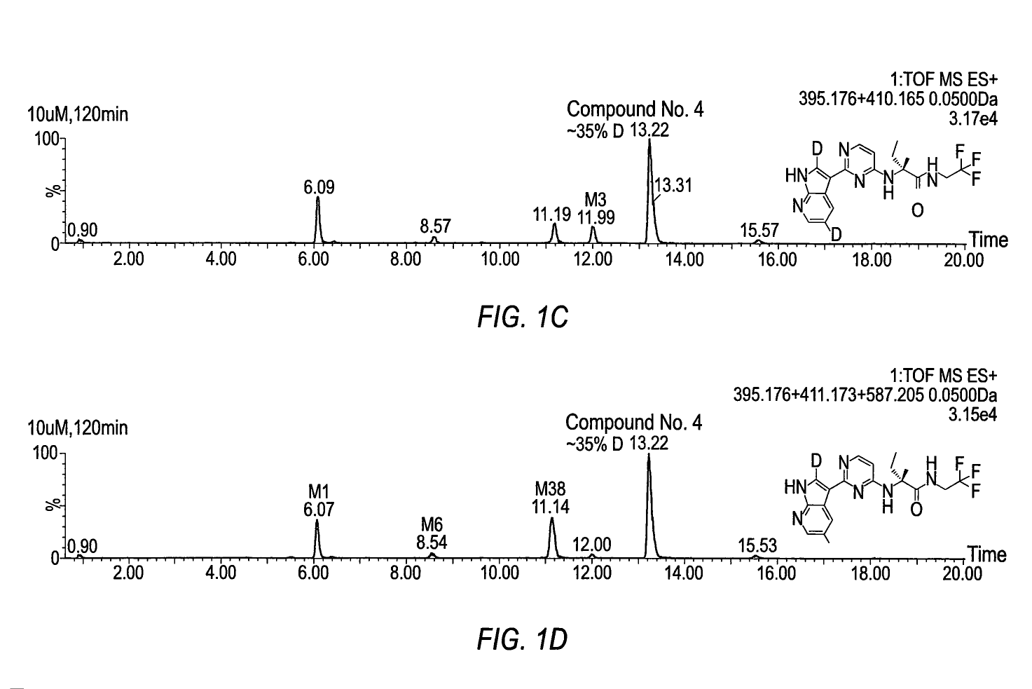

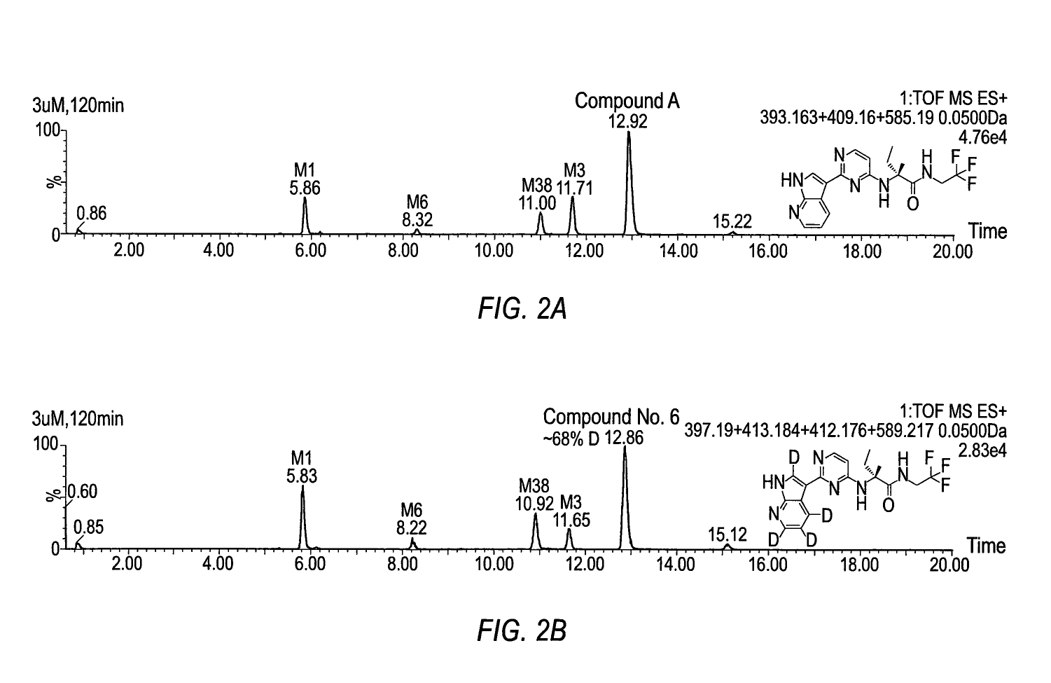

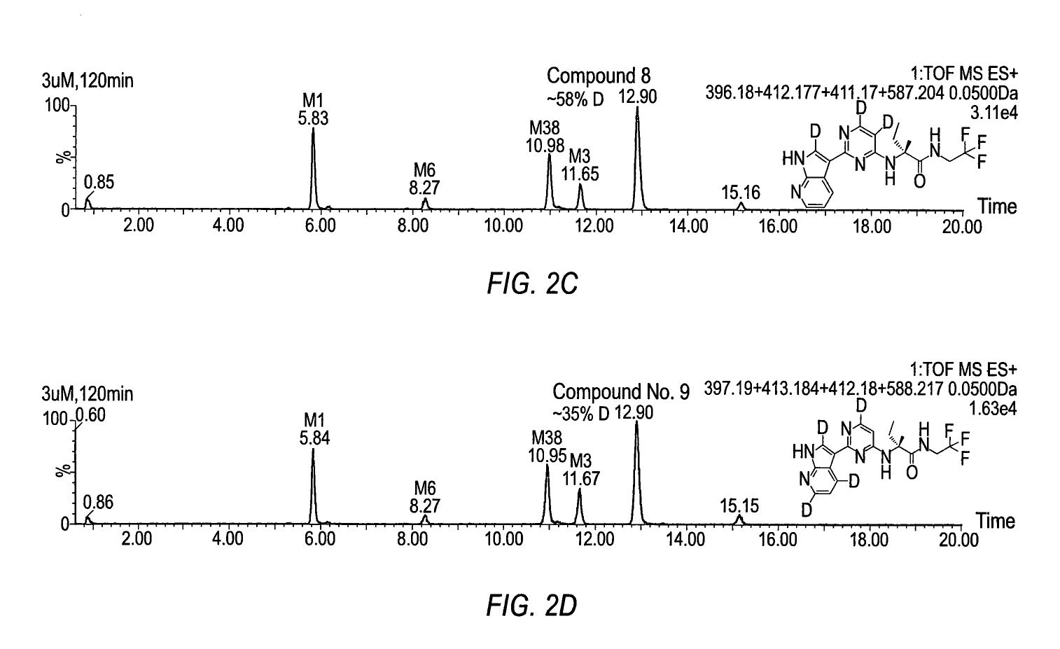

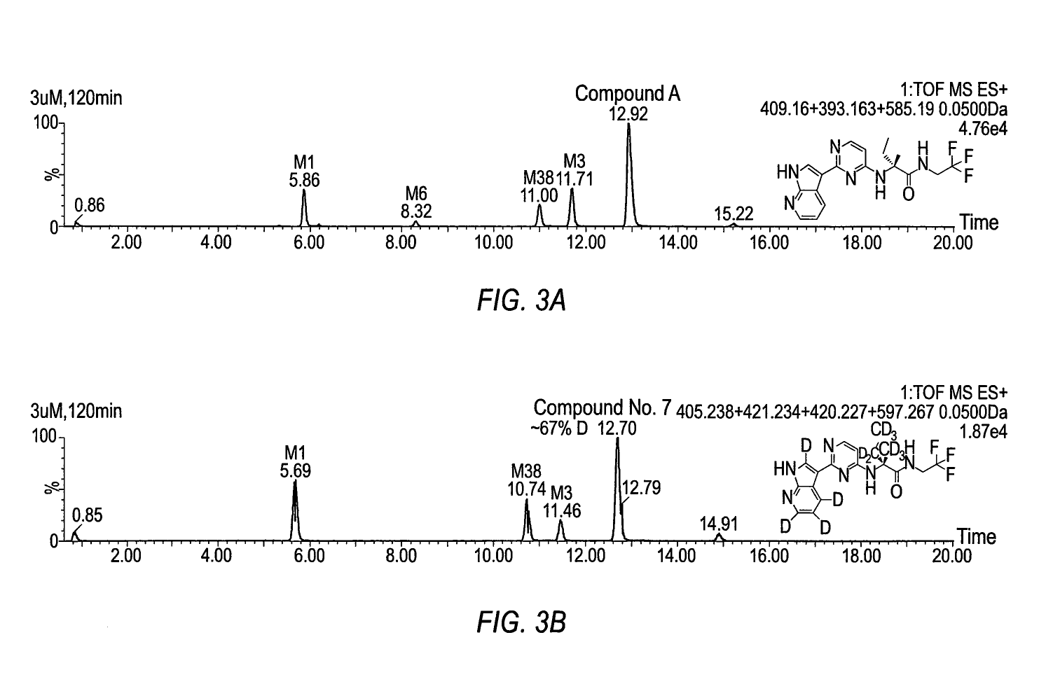

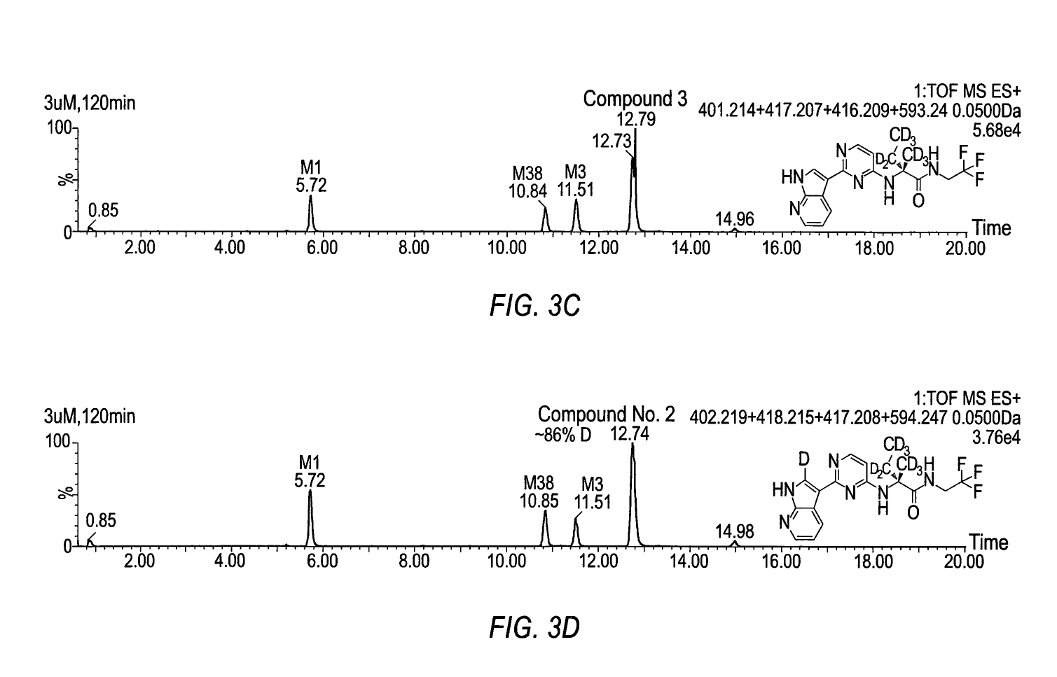

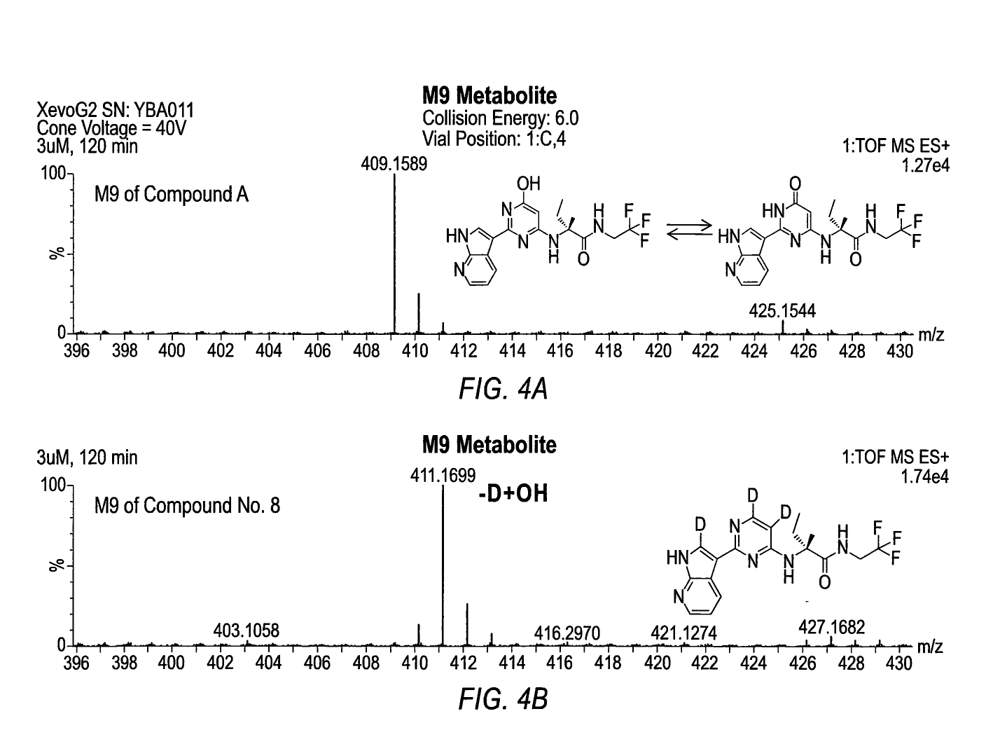

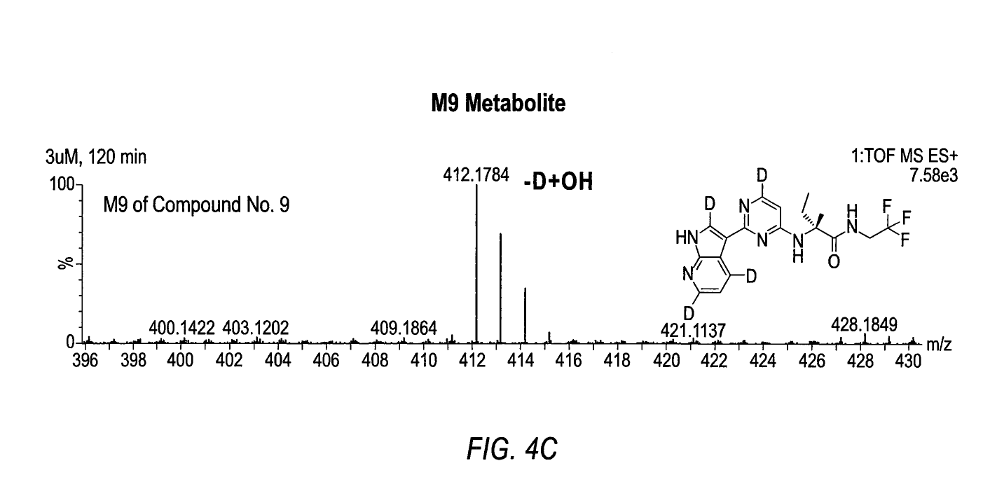

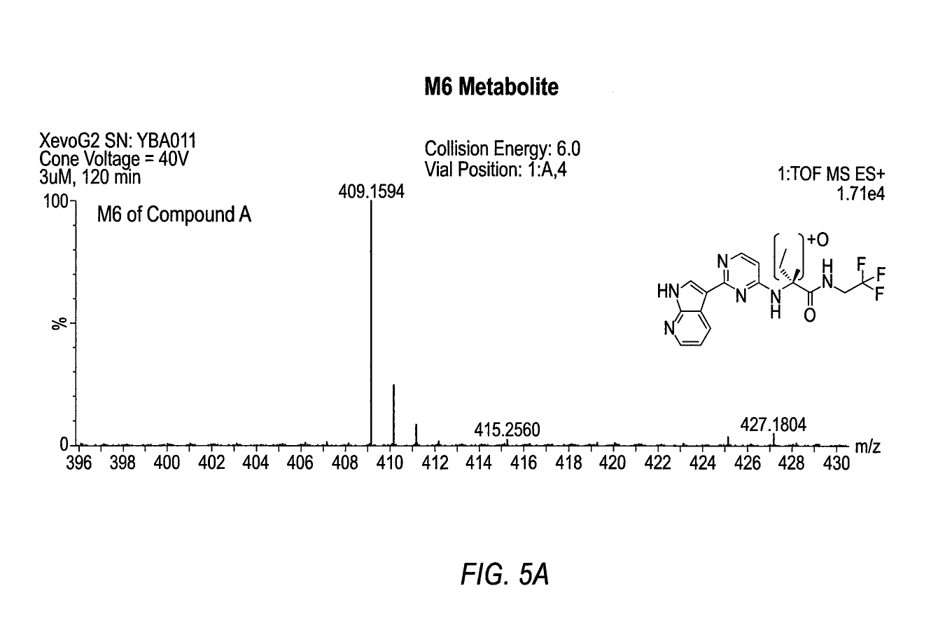

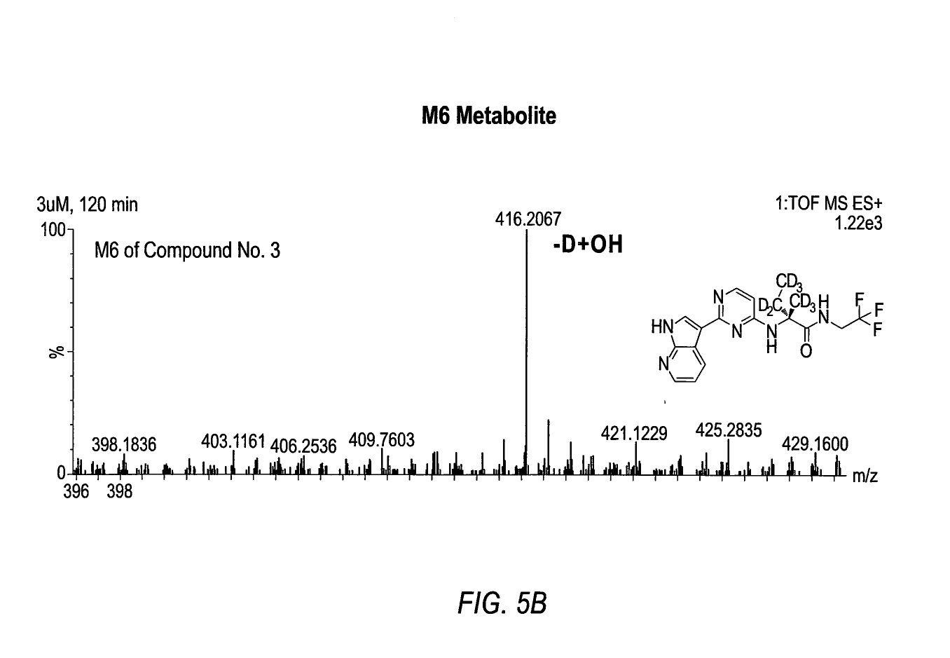

Referring to FIGS. 1A-5B, the data from these assessments indicates that no new metabolites were observed for Compound 1-a in human hepatocytes. The metabolite profile of Compound 1-a was similar to that of Compound A in human hepatocytes. Furthermore, the kinetic isotope effect was only observed for the formation of the M3 and M6 metabolites of Compound A in human hepatocytes, noting that the M6 metabolite is a minor metabolite.

### Example 7

### Assessment of the Effect of Deuterating Compound A at the C2 Position of the Azaindole Ring System.

Incubation Details

Cryopreserved Human Hepatocytes lot TFF (purchased from Celsis) were used. Hepatocytes were thawed using CHRM and suspended in Williams E media containing cell maintenance supplement package. 1000 μL of a final cell concentration 1 million cells/mL were placed in individual incubation wells (24-well plate set-up). Incubation was conducted at 37° C. and kept in a CO₂/O₂ humidified incubator. 10 μL of compound stock (1, 10, or 100 μM) were spiked into cell matrix to achieve final incubation concentrations of 0.01, 0.1, and 1 μM. Matrix was swirled prior to the removal of each time-point and 50 μL of sample were removed and added to 200 μL of acetonitrile containing internal standard, IS. Time-points were sampled at 0, 15, 30, 60, and 120 minutes.

Bioanlysis Details

Standards and QCs of Compound A and Compound B, and of Compound 1-a and Compound B were prepared from 0.01 μM to 20 μM in 95/5 H₂O/ACN in a glass-coated deep-well plate. 1 μL standard/QC was added to 90 μL matrix (final concentration range of 0.001 μM to 2 μM), and added to 400 μL IS, then vortexed and centrifuged at 3700 RPM for 30 minutes. 150 pi, aliquot of supernatant was transferred to a 96 shallow-well plate, evaporated to dryness, and reconstituted with 50 μL 95/5 H₂O/ACN. Samples were analyzed by MRM on an ABSciex API5500-QTrap paired with an Agilent 1290 UPLC and a CTC PAL autosampler. A 6-minute gradient method using a HALO C18 2.1×50 mm 2.7 μm column made by Advanced Materials Technology was used for the analysis.

TABLE B
Gradient Table.

| Time (min) | Flow (μL/min) | % A | % B |
|---|---|---|---|
| 0.00 | 600 | 90 | 10 |
| 3.00 | 600 | 74 | 26 |
| 3.10 | 600 | 5 | 95 |
| 4.40 | 600 | 5 | 95 |
| 4.50 | 600 | 90 | 10 |
| 6.00 | 600 | 90 | 10 |

TABLE C
LC-MS MRM Transitions

| Compound | Precursor | Product | DP | CE |
|---|---|---|---|---|
| IS | 406.2 | 346.2 | 60 | 20 |
| Compound A | 393.1 | 212.0 | 70 | 25 |
| Compound 1-a | 394.1 | 213.1 | 70 | 25 |
| Compound B | 409.1 | 282.0 | 70 | 25 |

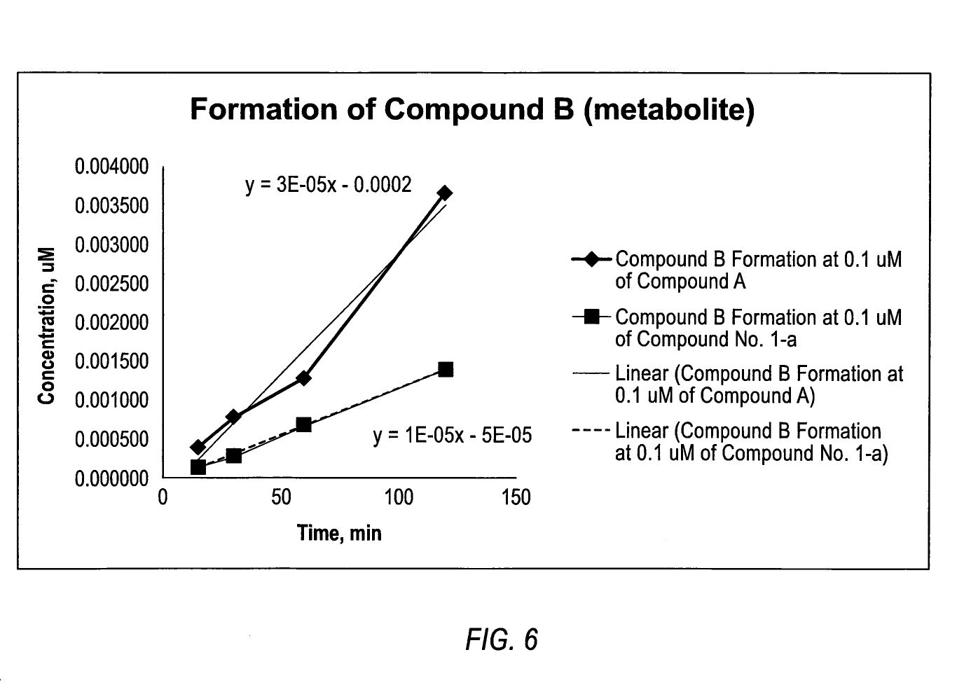

Referring to FIG. 6, based on the examination of the rate of formation of the Compound B (the metabolite), Compound 1-a slows formation of Compound B by 2.5 fold when compared to the rate of formation of Compound B with Compound A (the native compound).

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

What is claimed is:

1. A compound having the structure: 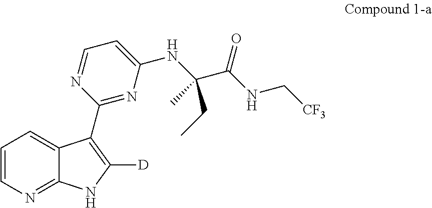 [SMILES: [2H]c1[nH]c2ncccc2c1-c1nccc(N[C@](C)(CC)C(=O)NCC(F)(F)F)n1]
or a pharmaceutically acceptable salt thereof.

2. A compound of Formula II-a: 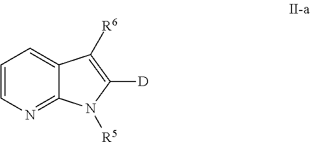
or a pharmaceutically acceptable salt thereof, wherein
R⁵ is —H or —PG¹, wherein PG¹ is an amine protecting group; and
R⁶ is —H, halo, or —B(OR⁷)₂, wherein each R⁷ is independently —H, C₁₋₄ alkyl, or two —OR⁷ groups taken together with the boron atom to which they are attached form a 5-6 membered heterocycle optionally substituted with 1-4 C₁₋₃ alkyl groups.

3. The compound according to claim 2, wherein R⁵ is —PG¹, and —PG¹ is —SO₂-phenyl or Boc, wherein the phenyl is optionally substituted with alkyl.

4. The compound according to claim 3, wherein the phenyl is unsubstituted.

5. The compound according to claim 2, wherein R⁶ is halo or —B(OR⁷)₂.

6. The compound according to claim 5, wherein R⁶ is halo.

7. The compound according to claim 6, wherein R⁶ is —Cl or —Br.

8. The compound according to claim 7, wherein R⁶ is Br.

9. The compound according to claim 5, wherein R⁶ is —B(OR⁷)₂, and each R⁷ is hydrogen.

10. The compound according to claim 2, wherein the compound of Formula II has the structure:

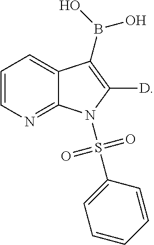

11. A compound of Formula III-a: 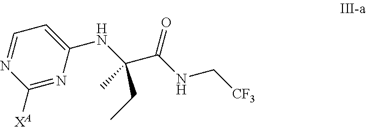
or a pharmaceutically acceptable salt thereof, wherein each X^{A} is a leaving group.

12. The compound according to claim 11, wherein X^{A} is halo.

13. The compound according to claim 12, wherein X^{A} is —Cl or —Br.

14. The compound according to claim 11, wherein the compound of Formula III-a has the structure:

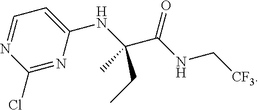 [SMILES: CC[C@@](C)(Nc1ccnc(Cl)n1)C(=O)NCC(F)(F)F]

15. A process for preparing Compound 1-a: 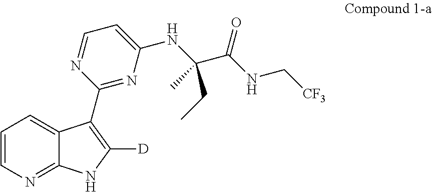 [SMILES: [2H]c1[nH]c2ncccc2c1-c1nccc(N[C@](C)(CC)C(=O)NCC(F)(F)F)n1]
or a pharmaceutically acceptable salt thereof, comprising the steps of:
a-2) reacting a compound of Formula 1-1a, wherein each R⁷ is independently —H, —C₁₋₄ alkyl, or two —OR⁷ groups taken together with the boron atom to which they are attached form a 5-6 membered heterocycle optionally substituted with 1-4 C₁₋₃ alkyl groups, and PG¹ is an amine protecting group, with a compound of Formula III-a, wherein X^{A} is a leaving group, 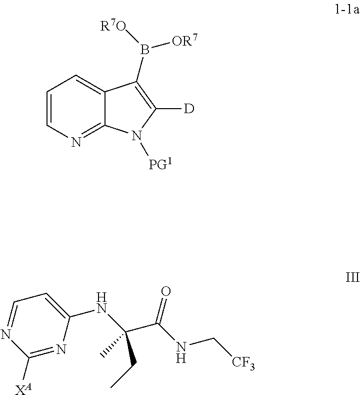
in the presence of a base and a palladium catalyst to generate a compound of Formula IV, and 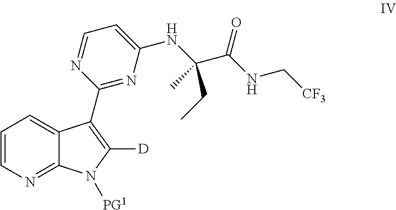
b-2) deprotecting the compound of Formula IV to generate Compound 1-a.

16. The process according to claim 15, wherein X^{A} is halo.

17. The process according to claim 15, wherein X^{A} is —Cl.

18. The process according to claim 15, wherein R⁷ is —H.

19. The process according to claim 15, wherein PG¹ is —SO₂-phenyl or Boc, wherein the phenyl is optionally substituted with alkyl.

20. The process according to claim 19, wherein the phenyl is unsubstituted.

21. The process according to claim 15, further comprising:
c-2) reacting a compound of Formula 4-2: 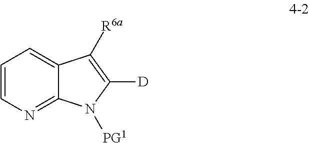
wherein R^{6a} is a leaving group, with a borylating agent to generate the compound of Formula 1-1a.

22. The process according to claim 21, wherein the borylating agent comprises bis-pinacol borane.

23. The process according to claim 21, wherein the borylaying agent comprises 2-isopropoxy-4,4,5,5-tetramethyl-1,3,2-dioxaborolane.

24. The process according to claim 21, further comprising:
d-2) reacting a compound of Formula VI: 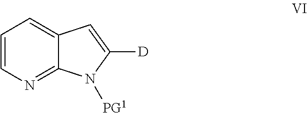
with R^{6a}—X^{B}, wherein X^{B} is halo, in the presence of an organic solvent to generate the compound of Formula 4-2.

25. The process according to claim 24, wherein R^{6a}—X^{B} is Br₂.

26. The process according to claim 24, further comprising:
e) protecting the compound of Formula 6: 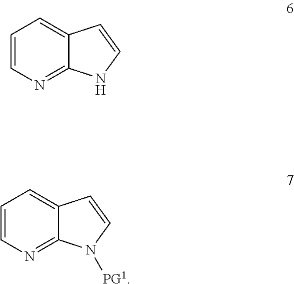
with an amine protecting group POI, to generate the compound of Formula 7; and
reacting the compound of Formula 7 with a deuterating agent to generate the compound of Formula VI.
